(19) United States
(12) Patent Application Publication
(10) Pub. No.: US 2024/0358777 A1
(43) Pub. Date: Oct. 31, 2024

(54) COMPOSITIONS COMPRISING BACILLUS AMYLOLIQUEFACIENS FOR PREVENTING AND TREATING ULCERATIVE COLITIS

(71) Applicant: Artugen Therapeutics Ltd., Dublin (IE)

(72) Inventors: Ronald Farquhar, Boston, MA (US); Susan L. Acton, Lexington, MA (US); Laurent Chesnel, Sant Antonin Noble Val (FR)

(73) Assignee: Artugen Therapeutics Ltd., Dublin (IE)

(21) Appl. No.: 18/688,067

(22) PCT Filed: Sep. 6, 2022
(86) PCT No.: PCT/IB2022/058383

§ 371 Date: Feb. 29, 2024

Related U.S. Application Data
(60) Provisional application No. 63/241,439, filed on Sep. 7, 2021.

(51) Int. Cl. A61K 35/742; A61K 31/196; A61K 31/436; A61K 31/519; A61K 31/52; A61K 31/541; A61K 31/573; A61K 31/58; A61K 31/655; A61K 35/00; A61K 38/13; A61K 39/395; A61P 1/04; A61P 3/04

(52) U.S. Cl. CPC A61K 35/742; A61K 31/196; A61K 31/436; A61K 31/519; A61K 31/52; A61K 31/541; A61K 31/573; A61K 31/58; A61K 31/655; A61K 38/13; A61K 39/3955; A61P 1/04; A61P 3/04; A61K 2035/11

(57) ABSTRACT

Disclosed herein are compositions and methods for preventing, ameliorating, or treating ulcerative colitis and/or reducing the severity of one or more risk factors, signs, or symptoms associated with ulcerative colitis. In particular, the technology of the present disclosure relates to methods for administering an effective amount of a composition comprising a strain of an operational group Bacillus amyloliquefaciens bacteria identified as ART24 to a subject suffering from or at risk for ulcerative colitis.

### CROSS-REFERENCE TO RELATED APPLICATION

The present application claims priority to U.S. Provisional Patent Application No. 63/241,439, filed on Sep. 7, 2021, the contents of which are hereby incorporated by reference in their entirety.

### TECHNICAL FIELD

The present technology relates to methods and compositions for preventing, ameliorating, or treating ulcerative colitis and/or reducing the severity of one or more risk factors, signs, or symptoms associated with ulcerative colitis. In particular, the present technology relates to administering an effective amount of a composition comprising a strain of an operational group Bacillus amyloliquefaciens bacteria, identified as ART24, to a subject suffering from or at risk for ulcerative colitis.

### BACKGROUND

The following description is provided to assist the understanding of the reader. None of the information provided or references cited is admitted to be prior art to the compositions and methods disclosed herein.

Ulcerative colitis (UC) is a chronic disease with recurrent uncontrolled inflammation of the colon. The rectum is always affected with inflammation spreading from the distal to the proximal colonic segments. Ulcerative colitis affects the innermost lining of the colon and rectum, with symptoms usually developing gradually. Ulcerative colitis can be debilitating and lead to life-threatening complications. Accordingly, there is a need to develop approaches for the treatment and prevention of ulcerative colitis.

### SUMMARY

In one aspect, the disclosure of the present technology provides a method for treating or preventing ulcerative colitis in a subject in need thereof, the method comprising administering to the subject a therapeutically effective amount of a pharmaceutical composition comprising bacterial strain ART24 (NCIMB Accession No. 43088).

In some embodiments, the ulcerative colitis is ulcerative proctitis, proctosigmoiditis, left-sided colitis, or pancolitis.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

In some embodiments, the pharmaceutical composition is administered enterally.

In some embodiments, the method further comprises separately, sequentially, or simultaneously administering one or more additional agents to the subject. In some embodiments, the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

In some embodiments, the subject is a human.

In some embodiments, the treatment comprises one or more of a reduction in weight loss, an improvement in disease activity index (DAI) scores, or reduced colonic levels of inflammatory markers. In some embodiments, the colonic inflammatory markers comprise one or more of IL-1β, IL-6, TNF-α, or myeloperoxidase (MPO).

In another aspect, the disclosure of the present technology provides the use of a composition in the preparation of a medicament for treating or preventing ulcerative colitis in a subject in need thereof, wherein the composition comprises bacterial strain ART24 (NCIMB Accession No. 43088.

In some embodiments, the ulcerative colitis is ulcerative proctitis, proctosigmoiditis, left-sided colitis, or pancolitis.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

In some embodiments, the pharmaceutical composition is formulated for enteral administration.

In some embodiments, the use further comprises separately, sequentially, or simultaneously administering one or more additional agents to the subject. In some embodiments, the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

In some embodiments, the subject is a human.

In some embodiments, the treatment comprises one or more of a reduction in weight loss, an improvement in disease activity index (DAI) scores, or reduced colonic levels of inflammatory markers. In some embodiments, the colonic inflammatory markers comprise one or more of IL-1β, IL-6, TNF-α, or myeloperoxidase (MPO).

In another aspect, the disclosure of the present technology provides a composition used for treating or preventing ulcerative colitis in a subject in need thereof, wherein the composition comprises bacterial strain ART24 (NCIMB Accession No. 43088.

In some embodiments, the ulcerative colitis is ulcerative proctitis, proctosigmoiditis, left-sided colitis, or pancolitis.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

In some embodiments, the pharmaceutical composition is formulated for enteral administration.

In some embodiments, the use further comprises separately, sequentially, or simultaneously administering one or more additional agents to the subject. In some embodiments, the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

In some embodiments, the subject is a human.

In some embodiments, the treatment comprises one or more of a reduction in weight loss, an improvement in disease activity index (DAI) scores, or reduced colonic levels of inflammatory markers. In some embodiments, the colonic inflammatory markers comprise one or more of IL-1β, IL-6, TNF-α, or myeloperoxidase (MPO).

### BRIEF DESCRIPTION OF THE DRAWINGS

FIG. 1A is a schematic depicting the experimental design for the ulcerative colitis-like DSS murine model.

FIGS. 1B and 1C are charts showing the mean percent daily weight change (plus or minus the standard error) in each group of mice over the course of the experiment. “Cryoprotectant alone”=trehalose.

FIGS. 1D and 1E are charts showing the composite Disease Activity Index (DAI) score (plus or minus the standard error) in each group of mice over the course of the experiment. “Cryoprotectant alone”=trehalose.

FIG. 1F is a chart showing the average level of colonic IL-1β in mice from groups 1, 2, 4, and 5 at the end of the experiment (Day 19). Upon euthanasia, colon tissue homogenates were prepared from the Day 19 samples. The colon tissue supernatant was used to determine the protein levels of IL-1β in Groups 1, 2, 4, and 5. The mean colon tissue cytokines concentrations±SEM are shown. *p≤0.05. Statistical significance was determined by one-way ANOVA followed by Dunnett's post test for multiple comparisons vs Group 2.

FIG. 1G is a chart showing the average level of colonic IL-6 in mice from groups 1, 2, 4, and 5 at the end of the experiment (Day 19). Upon euthanasia, colon tissue homogenates were prepared from the Day 19 samples. The colon tissue supernatant was used to determine the protein levels of IL-6 in Groups 1, 2, 4, and 5. The mean colon tissue cytokines concentrations±SEM are shown. *p≤0.05. Statistical significance was determined by one-way ANOVA followed by Dunnett's post test for multiple comparisons vs Group 2.

FIG. 1H is a chart showing the average level of colonic IL-10 in mice from groups 1, 2, 4, and 5 at the end of the experiment (Day 19). Upon euthanasia, colon tissue homogenates were prepared from the Day 19 samples. The colon tissue supernatant was used to determine the protein levels of IL-1β in Groups 1, 2, 4, and 5. The mean colon tissue cytokines concentrations±SEM are shown. *p≤0.05. Statistical significance was determined by one-way ANOVA followed by Dunnett's post test for multiple comparisons vs Group 2.

FIG. 1I is a chart showing the average level of colonic TNF-α in mice from groups 1, 2, 4, and 5 at the end of the experiment (Day 19). Upon euthanasia, colon tissue homogenates were prepared from the Day 19 samples. The colon tissue supernatant was used to determine the protein levels of TNF-α in Groups 1, 2, 4, and 5. The mean colon tissue cytokines concentrations±SEM are shown. *p≤0.05. Statistical significance was determined by one-way ANOVA followed by Dunnett's post test for multiple comparisons vs Group 2.

FIG. 1J is a chart showing the average level of colonic myeloperoxidase (MPO) in mice from groups 1, 2, 4, and 5 at the end of the experiment (Day 19). Upon euthanasia, colon tissue homogenates were prepared from the Day 19 samples. The colon tissue supernatant was used to determine the protein levels of MPO in Groups 1, 2, 4, and 5. The mean colon tissue MPO concentrations±SEM are shown. *p≤0.05. Statistical significance was determined by one-way ANOVA followed by Dunnett's post test for multiple comparisons vs Group 2.

FIG. 1K is a chart showing mouse distal colon sum colitis scores. Group mean plus or minus standard error. Treatment with ART24 in either form (Groups 4 and 5) was associated with lowered sum scores compared to vehicle treatment.

FIG. 1L is a chart showing mouse distal colon mean histopathology scores. Group mean plus or minus standard error. Reductions in sum scores with ART24 treatment were primarily driven by lowered inflammation, gland loss, and hyperplasia scores in the distal colon. 1=Group 1; 2=Group 2; 4=Group 4; 5=Group 5.

FIG. 1M is a chart showing mouse distal colon mean immunolabeling scores. Group mean plus or minus standard error. Loss of tight junction expression in the distal colon was seen with DSS administration. 1=Group 1; 2=Group 2; 4=Group 4; 5=Group 5.

FIG. 1N is group 1 (No DSS), Animal 1 Distal Colon. Hematoxylin and eosin stain, 100×. Non-lesioned colon is captured. The mucosa (M), artifactually separated submucosa (SM), and the tunica muscularis externa (TME) are indicated.

FIG. 1O is Group 2 (3% DSS+Vehicle), Animal 10 Distal Colon. Hematoxylin and eosin stain, 100×. A regionally extensive area of gland loss with infiltration of neutrophils, lymphocytes, and macrophages (**) is overlain by eroded surface epithelium (black arrows demarcate erosion), with luminal exudation of neutrophils (N). The adjacent mucosal glands (M) are hyperplastic, with gland elongation, crypt branching, reduced goblet cells, and increased mitotic figures observed. Inflammatory cells (*) also extend into the submucosa (SM). The tunica muscularis externa (TME) is indicated.

FIG. 1P is Group 4 (3% DSS+ART24, 5×108/dose), Animal 34 Distal Colon. Hematoxylin and eosin stain, 100×. Although multiple areas of colonic gland loss and inflammatory cell infiltration (**) are captured, the surface epithelium is largely intact. Adjacent mucosal glands (M) are minimally or non-hyperplastic. The submucosa (SM) contains low numbers of infiltrating mononuclear inflammatory cells (*). The tunica muscularis externa (TME) is indicated.

FIG. 1Q is Group 5 (3% DSS+ART24 [Washed], 5×108/dose), Animal 52 Distal Colon. Hematoxylin and eosin stain, 100×. Similar to the other ART24-treated sample (Group 4, Image 3), areas of inflammatory cell infiltration and gland necrosis/loss (**) are overlain by intact surface epithelium. Modest hyperplasia is observed in the mucosal glands (M) adjacent areas of loss. The submucosa (SM) and the tunica muscularis externa (TME) are indicated.

FIG. 1R is Group 1 (No DSS), Animal 1 Distal Colon. ZO-1 routine immunohistochemistry, 100×. Contiguous apical ZO-1 immunolabeling (brown staining) is seen on the mucosal surface (blue arrowheads) and lining well oriented glands (black arrowheads). Note that in mucosal glands (M) that are not well captured, the apical glandular immunolabeling is not visible. The submucosa (SM) is indicated. ZO-1 immunolabeling is also seen in the muscularis mucosae, blood vessels, and the tunica muscularis externa (TME), but this immunolabeling was not considered for scoring.

FIG. 1S is Group 2 (3% DSS+Vehicle), Animal 8 Distal Colon. ZO-1 routine immunohistochemistry, 100×. An area of mucosal (M) epithelial hyperplasia adjacent a zone of inflammation, gland loss (**), and erosion (E) is captured. Most surface (blue arrowhead) and gland luminal (black arrowhead) ZO-1 immunolabeling is intact but a few areas of reduced luminal immunolabeling (black arrow) and surface immunolabeling discontinuity (blue arrow) are observed. The submucosa (SM) and the tunica muscularis externa (TME) are indicated.

FIG. 1T is Group 4 (3% DSS+ART24, 5×108/dose), Animal 34 Distal Colon. ZO-1 routine immunohistochemistry, 100×. In a region of partial gland loss and inflammatory cell infiltration (**) the majority of mucosal (M) ZO-1 immunolabeling is intact, including along the epithelial surface (blue arrowhead) and gland lumina (black arrowheads). Very slight loss of ZO-1 labeling/discontinuity of labeling is focally seen within the surface epithelium (blue arrow). The submucosa (SM) and the tunica muscularis externa (TME) are indicated.

FIG. 1U is Group 5 (3% DSS+ART24 [Washed], 5×108/dose), Animal 51 Distal Colon. ZO-1 routine immunohistochemistry, 100×. A small area of gland loss and inflammatory cell infiltration (**) is bordered by intact mucosal glands (M). The surface epithelial (blue arrowhead) and glandular lumina (black arrowhead) apical labeling typical of ZO-1 is retained for the most part. Small regions of reduced surface (blue arrow) and gland lumina (black arrow) immunolabeling are present. The submucosa (SM) and the tunica muscularis externa (TME) are indicated.

FIG. 1V is Group 1 (No DSS), Animal 1 Distal Colon. Occludin routine immunohistochemistry, 100×. Dense immunolabeling for occludin (brown staining) is seen in a membranous pattern along intercellular borders on the mucosal surface (blue arrowheads) and within glands (black arrowheads), although the latter is a little more difficult to identify due to the presence of goblet cells. Mucosal glands (M), the submucosa (SM), and the tunica muscularis externa (TME) are indicated.

FIG. 1W is Group 2 (3% DSS+Vehicle), Animal 7 Distal Colon. Occludin routine immunohistochemistry, 100×. Areas of remaining mucosal glands (M) exhibit reduced overall immunolabeling for occludin. Reduced to absent surface (blue arrow) and glandular (black arrows) immunolabeling is observed. Areas of gland loss and inflammatory cell infiltration (**), more normal occludin immunolabeling (surface: blue arrowhead; glandular: black arrowhead), the submucosa (SM), and the tunica muscularis externa (TME) are indicated.

FIG. 1X is Group 4 (3% DSS+ART24, 5×108/dose), Animal 35 Distal Colon. Occludin routine immunohistochemistry, 100×. Areas of gland loss/inflammation (**) and surface epithelial loss (erosion: E; note the lack of occludin labeling) are captured, with some intact mucosal glands (M) remaining. Within those glands, immunolabeling is largely retained (surface: blue arrowhead; glandular: black arrowhead), with a few areas of slightly reduced surface (blue arrow) and luminal (black arrow) immunolabeling seen. The submucosa (SM) and the tunica muscularis externa (TME) are indicated.

FIG. 1Y is Group 5 (3% DSS+ART24 [Washed], 5×108/dose), Animal 55 Distal Colon. Occludin routine immunohistochemistry, 100×. Bordering an area of gland loss and inflammation (**) with erosion (E), mucosal glands (M) exhibit slightly reduced luminal (black arrow) and surface (blue arrow) occludin immunolabeling. Areas with unaffected labeling are also observed (luminal: black arrowhead, surface: blue arrowhead). The submucosa (SM) is indicated.

FIG. 2 is a chart showing the effect of simulated gastric fluid (pH 3) on ART24 spores.

FIG. 3A is schematic showing the details of the colon tissue collection.

FIG. 3B is a chart showing body weight change in animals over the course of the study. Animals were weighed daily and body weight change as compared to Day 0 was calculated. The AUC was calculated using the trapezoidal rule transformation. Data is presented as mean±SEM. n=6-20 per group.

FIG. 3C is a chart showing the body weight AUC. Animals were weighed daily and body weight change as compared to Day 0 was calculated. The AUC was calculated using the trapezoidal rule transformation. Statistical significance between groups was determined by one-way ANOVA with Dunnett's multiple comparisons test used to compare all groups to the diseased vehicle-control group. *p<0.05; ****p<0.0001. Data is presented as mean±SEM. n=6-20 per group.

FIG. 3D is a chart showing composite DAI scores for each of Groups 1-7. Animals were observed daily for weight loss, diarrhea, and blood in stool. Each of these parameters was individually scored according to a DAI scoring scheme.

FIG. 3E is a chart showing the AUC for the composite DAI scores for each of Groups 1-7. Statistical significance between groups was determined by one-way ANOVA with Dunnett's multiple comparisons test used to compare al groups to the diseased vehicle-control group. Data is presented as mean±SEM. **p<0.01, ****p<0.0001. n=6-20 per group.

FIG. 3F shows representative endoscopy images from Groups 1-4 at Day 12. n=6-20 per group.

FIG. 3G shows representative endoscopy images from Groups 5-7 at Day 12. n=6-20 per group.

FIG. 3H is a chart showing colon measurements at euthanasia. Animals were euthanized on Day 12. The colon was excised, measured, and weighed, and the colon weight:length ratio was calculated. Statistically significant differences between groups were assessed by one-way ANOVA with Holm-Sidak's multiple comparison post-test used to compare all groups to Group 2 (vehicle and DSS vs. treatments). ***p<0.001, ****p<0.0001. Data is presented as mean±SEM. n=6-20 per group.

FIG. 3I is a chart showing colon myeloperoxidase measurements. Animals were euthanized on Day 12 and colon tissue was collected and processed for immunoassay. Data is presented for individual animals, with mean±SEM. n=6-20 per group.

FIGS. 3J-3M are charts showing colon tissue homogenate IL-1β, IL-6, IL-10, and TNFα levels on Day 12. Colon portions were homogenized and analyzed for protein levels of mouse inflammatory mediators. Samples that were out-of-range on the low end of the curve were entered as 50% of the lowest standard; if on the high end of the curve, they were entered as the highest standard curve. Statistically significant differences between groups were assessed by one-way ANOVA with Holm-Sidak's multiple comparison post-test used to compare all groups to Group 2 (vehicle and DSS vs. treatments). Data is presented for individual animals, with mean±SEM. *p<0.05. n=6-20 per group.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “administration” of an agent, drug, bacterial strain or spore thereof, or composition of the present technology to a subject includes any route of introducing or delivering to a subject a compound to perform its intended function. Administration can be carried out by any suitable route, including orally, intranasally, parenterally (intravenously, intramuscularly, intraperitoneally, or subcutaneously), topically, or by inhalation. In some embodiments, the compositions of the present technology are formulated for enteric administration. In some embodiments, the compositions are formulated for oral, sublingual, or rectal delivery. In some embodiments, the compositions are formulated for use as a probiotic. In some embodiments, the compositions are formulated for use as a live biotherapeutic. As used herein, administration includes self-administration and administration by another.

As used herein, “ART24” refers to a bacterial strain, or spore thereof, having been deposited under NCIMB Accession No. 43088, or compositions comprising the strain. ART24 is considered a member of an “operational group B. amyloliquefaciens” that comprises the soil-borne B. amyloliquefaciens, and plant associated Bacillus siamensis and Bacillus velezensis.

As used herein, the term “dietary supplement” refers to a product intended to supplement the diet. Typically, a dietary supplement is a product that is labeled as a dietary supplement and is not represented for use as a conventional food or as a sole item of a meal or the diet. A dietary supplement can be consumed by a subject independent of any food, unlike a food additive, which is incorporated into a food or food composition during the processing, manufacture, preparation, or delivery of the food or food composition, or just prior to its consumption.

As used herein, the “disease activity index” or “DAI” refers to a metric used to standardize the severity of a subject's ulcerative colitis symptoms. The DAI is used to determine initial disease severity, change in activity over time, and response to treatment. The DAI provides a universal metric to encapsulate disease severity at a given time in a single number. Measurements may comprise, and are not limited to, the subject's weight, diarrhea, blood in stool, and activity level. For human subjects, in particular, measurements may comprise, and are not limited to stool frequency, rectal bleeding, mucosal appearance at endoscopy, and physician rating of disease.

As used herein, the term “edible product” refers to any substance, whether processed, semi-processed, or raw, which is intended for consumption by animals including humans. In some embodiments, the term “edible product” refers to a food that comprises the bacterial strain of the present technology. Any food to which the bacterial strain of the present technology is added is an edible product of the present technology. Any food in which a bacterial strain of the present technology is made to be present at a greater level is also an edible product of the present technology. Edible products comprising the bacterial strain of the present technology include dietary supplements, nutraceutical compositions, food additives, food compositions, food compositions in bulk, food additives in bulk, medical foods, and foods for special dietary use. Edible products of the present technology include, but are not limited to, fermented food products, soybean, soybean-based food products, fermented soybean, fermented soybean paste, mushroom, mung bean, locus bean, rice, or extracts thereof. In some embodiments, edible products of the present technology refer to a food that is formulated to be consumed or administered enterally to a subject.

As used herein, the terms “effective amount,” or “therapeutically effective amount,” and “pharmaceutically effective amount” refer to a quantity sufficient to achieve a desired therapeutic and/or prophylactic effect, e.g., an amount which results in the prevention of a disease, condition, and/or symptom(s) thereof. In the context of therapeutic or prophylactic applications, the amount of a composition administered to the subject will depend on the type and severity of the disease and on the characteristics of the subject, such as general health, age, sex, body weight, and tolerance to the composition drugs. It will also depend on the degree, severity, and type of disease or condition. The skilled artisan will be able to determine appropriate dosages depending on these and other factors. In some embodiments, multiple doses are administered. Additionally or alternatively, in some embodiments, multiple therapeutic compositions or compounds (e.g., pharmaceutical compositions comprising the bacterial strain alone or in combination with additional active agents, such as anti-inflammatory drugs, corticosteroids, and/or immunosuppressants) are administered. In the methods described herein, compositions comprising the bacterial strain of the present technology, or spores thereof, may be administered to a subject having one or more signs, symptoms, or risk factors of ulcerative colitis including, but not limited to, diarrhea with blood and/or mucous, abdominal pain and cramping, rectal pain, rectal bleeding, urgency to defecate, inability to defecate despite urgency, weight loss, tachycardia, anemia, bowel distension, fatigue, fever, and/or failure to grow (in children). For example, a “therapeutically effective amount” of the compositions of the present technology, includes levels at which the presence, frequency, or severity of one or more signs, symptoms, or risk factors of ulcerative colitis are, at a minimum, ameliorated. In some embodiments, a therapeutically effective amount reduces or ameliorates the physiological effects of ulcerative colitis, and/or the risk factors of ulcerative colitis, and/or the likelihood of developing ulcerative colitis. In some embodiments, a therapeutically effective amount is achieved by multiple administrations. In some embodiments, a therapeutically effective amount is achieved with a single administration.

As used herein, the term “food additive” refers to any substance not normally consumed as a food by itself and not normally used as a typical ingredient of the food, whether or not it has nutritive value, but which is intentionally added to food.

As used herein, the term “food for special dietary use” refers to a food that purports or is represented to be used for at least one of the following: supplying a special dietary need that exists by reason of physical, physiological, pathological of other condition.

As used herein, the terms “freeze-dried” or “freeze-drying” and “lyophilized” or “lyophilization” are used interchangeably and refer to a process that removes water from a product after it is frozen and placed under a vacuum and the products produced therefrom.

As used herein, the term “medical food” refers to a food that is formulated to be consumed or administered enterally under the supervision of a physician and which is intended for the specific dietary management of a disease or condition for which distinctive nutritional requirements are established.

As used herein, “pharmaceutically acceptable carrier and/or diluent” or “pharmaceutically acceptable excipient” includes but is not limited to solvents, dispersion media, coatings, antibacterial and antifungal agents, isotonic and absorption delaying agents, and the like. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch. The use of such media and agents for biologically active substances is well known in the art. Further details of excipients are provided below. Supplementary active ingredients, such as antimicrobials, for example antifungal agents, can also be incorporated into the compositions.

As used herein, “pharmaceutically acceptable excipient” refers to substances and compositions that do not produce an adverse, allergic, or other untoward reaction when administered to an animal or a human. As used herein, the term includes all inert, non-toxic, liquid or solid fillers, or diluents that do not react with the therapeutic substance of the invention in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol and/or certain oils.

As used herein, “probiotic” refers to bacteria comprising a component of the transient or endogenous flora of a subject administered to confer a beneficial prophylactic and/or therapeutic effect on the subject. In some embodiments, “probiotics” include “live biotherapeutic products.”

As used herein, “prevention,” “prevent,” or “preventing” of a disorder or condition refers to, in a statistical sample, reduction in the occurrence or recurrence of the disorder or condition in treated subjects/samples relative to an untreated controls, or refers delays the onset of one or more symptoms of the disorder or condition relative to the untreated controls.

As used herein “subject” and “patient” are used interchangeably. In some embodiments, the subject is an animal subject. In some embodiments, the animal subject is a mammal. In some embodiments, the mammalian subject is a human

As used herein, the term “simultaneous” administration refers to the administration of at least two agents by the same route and at the same time or at substantially the same time.

As used herein, the term “separate” administration refers to an administration of at least two agents at the same time or at substantially the same time by different routes.

As used herein, the term “sequential” administration refers to administration of at least two agents at different times, the administration route being identical or different. More particularly, sequential use refers to the whole administration of one agent before administration of the other agent(s) commences. It is thus possible to administer one of the agents over several minutes, hours, or days before administering another.

A “synergistic therapeutic effect” refers to a greater-than-additive therapeutic effect which is produced by a combination of at least two therapeutic agents, and which exceeds that which would otherwise result from the individual administration of the agents. For example, use of the bacterial strain of the present technology in conjunction with other agents for the treatment of ulcerative colitis may result in a greater than additive therapeutic effect.

In some embodiments, the synergistic effect may permit the use of lower doses of the bacterial strain of the present technology and/or other agents than would be required if each were used alone.

“Treating,” “treat,” “treated,” or “treatment” of a disease, condition, or disorder includes: (i) inhibiting the disease, condition, or disorder, i.e., arresting its development; (ii) relieving the disease, condition, or disorder, i.e., causing its regression; (iii) slowing progression of the disease, condition, or disorder; and/or (iv) inhibiting, relieving, or slowing progression of one or more symptoms of the disease, condition, or disorder.

It is to be appreciated that the various modes of treatment or prevention of medical diseases and conditions as described are intended to mean “substantial,” which includes total but also less than total treatment or prevention, and wherein some biologically or medically relevant result is achieved.

### II. Ulcerative Colitis

Ulcerative colitis is an inflammatory bowel disease that causes inflammation and ulcers in the digestive tract. Ulcerative colitis affects the innermost lining of the colon and rectum. Symptoms usually develop over time, rather than suddenly. Ulcerative colitis is often classified according to its location. Types of ulcerative colitis include: ulcerative proctitis, in which inflammation confined to the area closes to the rectum, and rectal bleeding may be the only sign of the disease; proctosigmoiditis, in which inflammation involves the rectum and sigmoid colon; left-sided colitis, in which inflammation extends from the rectum up through the sigmoid and descending colon; and pancolitis, which affects the entire colon and causes bouts of bloody diarrhea that may be severe, abdominal cramps and pain, fatigue, and significant weight loss).

Colitis can be caused by many things, such as infections from viruses or bacteria. Ulcerative colitis is particularly severe because it is not caused by infection and can be lifelong. The exact cause of ulcerative colitis is unknown. Anyone at any age, including young children, can develop ulcerative colitis. An individual may be at a higher risk of developing ulcerative colitis if: they have a close relative with inflammatory bowel disease; are between the ages of 15 and 30 years old, or older than 60 years of age; they are of Ashkenazi Jewish descent; they eat a high-fat diet; or if they frequently use nonsteroidal anti-inflammatory drugs (NSAIDS). A possible cause may include an autoimmune response leading to inflammation.

Although there is no universal standard for the diagnosis of ulcerative colitis, minimal diagnostic workup for UC includes medical history, clinical evaluation focusing on extraintestinal manifestations, full blood count, erythrocyte sedimentation rate (ESR), C-reactive protein (CRP), stool microbiology, ultrasound and endoscopy with mucosal biopsies. If there is any doubt about the diagnosis in the acute setting, endoscopic and histological confirmation should be repeated after a period of time has passed.

Traditionally, the choice of treatment for ulcerative colitis depends on the degree of activity, distribution, course of disease, frequency of relapses, extraintestinal manifestations, previous medications, side-effect profile, and the patient's individual wishes. Agents such as anti-inflammatory drugs are often the first step in the treatment of ulcerative colitis. These drugs include anti-inflammatory drugs in the 5-aminosalicylate family (e.g., sulfasalazine, mesalamine, balsalazide, and olsalazine), and corticosteroids (e.g., prednisone, budesonide). Additional agents for treatment may include: immunomodulators (e.g., 6-mercaptopurine, azathioprine, methotrexate); biologics such as anti-TNF-α drugs (e.g., infliximab, adalimumab, golimumab), integrin α4β7 inhibitors (e.g., vedolizumab), and IL-12/23 antibodies (e.g., ustekinumab); calcineurin inhibitors (e.g., cyclosporine, tacrolimus); Janus kinase (JAK) inhibitors (e.g., tofacitinib); ABT-494 (AbbVie); and filgotinib (GLPG0634, Galapagos and Gilead)). A final option for severe cases of UC is surgery to remove the colon and rectum (proctocolectomy).

In some embodiments, the present technology provides methods and compositions for the treating or preventing ulcerative colitis, including reducing the severity of one or more risk factors, signs, or symptoms associated with ulcerative colitis. In some embodiments the compositions comprise the ART24 strain, or spores thereof.

### III. ART24

The technology of the present disclosure relates to the use of bacteria strain ART24, or spores thereof, to treat or prevent ulcerative colitis. ART24 refers to a bacterial strain, or spore thereof, characterized by NCIMB Accession No. 43088, or compositions comprising the strain.

In some embodiments, the bacterial strain of the present technology, or spores thereof, is used in methods and compositions for treating or preventing ulcerative colitis. In some embodiments, the bacterial strain comprises a probiotic for preventing or controlling ulcerative colitis. In some embodiments, compositions of the present technology comprise vegetative bacterial cells. In some embodiments, compositions of the present technology comprise bacterial spores. In some embodiments, compositions of the present technology comprise a combination of vegetative bacterial cells and bacterial spores. In some embodiments, the compositions of the present technology comprise an ART24 bacterial strain that has been physically destructed or lysed. In some embodiments, the compositions of the present technology comprise an isolated fraction of lysed ART24 bacteria.

In some embodiments, the bacterial strain of the present technology, or spores thereof, is used in methods and edible product compositions for treating or preventing ulcerative colitis in a subject in need thereof. In some embodiments, the bacterial strain comprises a dietary supplement for preventing or controlling ulcerative colitis in a subject in need thereof.

### IV. Therapeutic and Prophylactic Methods

The following discussion is presented by way of example only, and is not intended to be limiting.

One aspect of the present technology includes methods of treating or preventing ulcerative colitis in a subject diagnosed as having, suspected as having, or at risk of having ulcerative colitis. In therapeutic applications, compositions or medicaments comprising the ART24 bacterial strain, or spore thereof, are administered to a subject suspected of, or already suffering from such a disease (such as, e.g., subjects exhibiting diarrhea with blood and/or mucous, abdominal pain and cramping, rectal pain, rectal bleeding, urgency to defecate, inability to defecate despite urgency, weight loss, tachycardia, anemia, bowel distension, fatigue, fever, and/or failure to grow (in children)), in an amount sufficient to cure, or at least partially arrest, the symptoms of the disease, including its complications and intermediate pathological phenotypes in development of the disease.

Subjects suffering from ulcerative colitis can be identified by any or a combination of diagnostic or prognostic assays known in the art. For example, typical symptoms of ulcerative colitis include, but are not limited to, diarrhea with blood and/or mucous, abdominal pain and cramping, rectal pain, rectal bleeding, urgency to defecate, inability to defecate despite urgency, weight loss, tachycardia, anemia, bowel distension, fatigue, fever, and/or failure to grow (in children).

In some embodiments, ulcerative colitis subjects treated with the bacterial strain of the present technology, or spores thereof, will show amelioration or elimination of one or more of the following symptoms: diarrhea with blood and/or mucous, abdominal pain and cramping, rectal pain, rectal bleeding, urgency to defecate, inability to defecate despite urgency, weight loss, tachycardia, anemia, bowel distension, fatigue, fever, and/or failure to grow (in children).

In one aspect, the present technology provides a method for preventing or delaying the onset of ulcerative colitis or symptoms of ulcerative colitis in a subject at risk of having ulcerative colitis. In some embodiments, the bacterial strain of the present technology is formulated as a probiotic useful as a food supplement. In some embodiments, the bacterial strain of the present technology is formulated as a live biotherapeutic product useful in pharmaceutical applications. In some embodiments, the bacterial strain of the present technology is formulated as a live biotherapeutic edible product useful in pharmaceutical applications.

### V. Modes of Administration and Effective Dosages

Compositions of the present technology for use in preventing, ameliorating, or treating ulcerative colitis and/or reducing the severity of one or more risk factors, signs, or symptoms associated with infection include live probiotic ART24 bacteria according to the present technology, provided in the form of vegetative cells and/or spores. In some embodiments, the bacterial strain is lyophilized. The compositions of the present technology are administered to the subject in effective amounts (i.e., amounts that have desired therapeutic effect). The dose and dosage regimen will depend upon the degree of the infection in the subject, the characteristics of the ART24 strain used, e.g., its therapeutic index, the subject, and the subject's history. The effective amount may be determined during pre-clinical trials and clinical trials by methods familiar to physicians and clinicians.

Compositions of the present technology may be formulated for adding to food, or used directly as a food supplement. The formulation may further include other probiotic agents or nutrients for promoting spore germination and/or bacterial growth.

Edible products of the present technology may be formulated for adding to food, or used directly as a dietary supplement. In some embodiments, the edible product comprises a fermented food product, soybean, mushroom, mung bean, locus bean, or rice. In some embodiments, the soybean is fermented soybean or fermented soybean paste. The formulation may further include other probiotic agents or nutrients for promoting spore germination and/or bacterial growth.

Additional components of the compositions of the present technology may include a preservative selected from the group consisting of sucrose, sodium ascorbate, and glutathione. In some embodiments the preservative is a cryoprotectant selected from the group consisting of a nucleotide, a disaccharide, a polyol, and a polysaccharide. In some embodiments, the cryoprotectant is selected from the group consisting of inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, skim milk, and cryoprotectant 18.

The ART24 bacterial strain described herein can be incorporated into pharmaceutical compositions for administration, singly or in combination, and given to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Carriers can be solid-based dry materials for formulations in powdered form, and can be liquid or gel-based materials for formulations in liquid or gel forms, which forms depend, in part, upon the routes or modes of administration.

In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch.

Pharmaceutical compositions are typically formulated to be compatible with its intended route of administration. Examples of routes of administration include enteric (e.g., oral, sublingual, rectal) administration. A therapeutic composition can be formulated to be suitable for oral administration in a variety of ways, for example in a liquid, a powdered food supplement, a solid food, a packaged food, a wafer, tablets, troches, or capsules, e.g., gelatin capsules, and the like. In some embodiments, the therapeutic compositions of the present technology comprise lyophilized ART24. In some embodiments, the lyophilized ART24 is encapsulated. A therapeutic composition can be formulated to be suitable for rectal administration in a variety of ways, for example in a suppository, liquid enema, or foam. Other formulations will be readily apparent to one skilled in the art. pH can be adjusted with acids or bases, such as hydrochloric acid or sodium hydroxide.

Additionally or alternatively, for edible products, pharmaceutical compositions of the present technology may be formulated as dietary supplements, nutraceutical compositions, food additives, food compositions, food compositions in bulk, food additives in bulk, medical foods, or foods for special dietary use, for enteric (e.g., oral, sublingual) administration.

Dosage, toxicity and therapeutic efficacy of any therapeutic agent can be determined by standard pharmaceutical procedures in cell cultures or experimental animals. The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately.

In some embodiments, the “edible products” such as whole soybeans and/or soybean pastes, are inoculated with a concentration of ART24 ranging from at least about 1×10² colony forming units (CFU)/mL to at least about 1×10¹⁰ CFU/mL, or any value in between. For example, in some embodiments, the edible products are inoculated with at least about 1×10³ CFU/mL to at least about 1×10¹⁰ CFU/mL ART24, at least about 1×10⁴ CFU/mL to at least about 1×10¹⁰ CFU/mL ART24, at least about 1×10⁵ CFU/mL to at least about 1×10¹⁰ CFU/mL ART24, at least about 1×10⁶ CFU/mL to at least about 1×10¹⁰ CFU/mL ART24, at least about 1×10⁷ CFU/mL to at least about 1×10¹⁰ CFU/mL ART24, at least about 1×10⁸ CFU/mL to at least about 1×10¹⁰ CFU/mL ART24, at least about 1×10⁹ CFU/mL to at least about 1×10¹⁰ CFU/mL ART24. In some embodiments, the edible products are inoculated with at least about 6×10⁸ CFU/mL ART24. In some embodiments, the edible products are inoculated with at least about 6×10⁹ CFU/mL ART24. In some embodiments, the edible products are inoculated with at least about 1×10⁴ CFU/mL ART24. In some embodiments, the edible products are inoculated with at least about 1×10⁴ CFU/mL ART24. In some embodiments, the edible products are inoculated with at least about 1×10⁶ CFU/mL ART24. In some embodiments, the edible products are inoculated with at least about 1×10⁸ CFU/mL ART24. In some embodiments, the edible products are inoculated with at least about 1×10⁹ CFU/mL ART24.

In some embodiments, the amount of ART24 in a pharmaceutical composition ranges from at least about 1×10² colony forming units (CFU) to at least about 1×10¹⁰ CFU, or any value in between. For example, in some embodiments, the pharmaceutical compositions comprise at least about 1×10³ CFU to at least about 1×10¹⁰ CFU ART24, at least about 1×10⁴ CFU to at least about 1×10¹⁰ CFU ART24, at least about 1×10⁵ CFU to at least about 1×10¹⁰ CFU ART24, at least about 1×10⁶ CFU to at least about 1×10¹⁰ CFU ART24, at least about 1×10⁷ CFU to at least about 1×10¹⁰ CFU ART24, at least about 1×10⁸ CFU to at least about 1×10¹⁰ CFU ART24, at least about 1×10⁹ CFU to at least about 1×10¹⁰ CFU ART24. In some embodiments, the pharmaceutical compositions comprise at least about 6×10⁸ CFU ART24. In some embodiments, the pharmaceutical compositions comprise at least about 6×10⁹ CFU ART24. In some embodiments, the pharmaceutical compositions comprise at least about 1×10⁴ CFU ART24. In some embodiments, the pharmaceutical compositions comprise at least about 1×10⁵ CFU ART24. In some embodiments, the pharmaceutical compositions comprise at least about 1×10⁶ CFU ART24. In some embodiments, the pharmaceutical compositions comprise at least about 1×10⁷ CFU ART24. In some embodiments, the pharmaceutical compositions comprise at least about 1×10⁸ CFU ART24. In some embodiments, the pharmaceutical compositions comprise at least about 1×10⁹ CFU ART24. In some embodiments, the amount of ART24 in a pharmaceutical composition ranges from at least about 5×10² CFU to at least about 5×10¹⁰ CFU, or any value in between. For example, in some embodiments, the pharmaceutical compositions comprise at least about 5×10³ CFU to at least about 5×10¹⁰ CFU ART24, at least about 5×10⁴ CFU to at least about 5×10¹⁰ CFU ART24, at least about 5×10⁵ CFU to at least about 5×10¹⁰ CFU ART24, at least about 5×10⁶ CFU to at least about 5×10¹⁰ CFU ART24, at least about 5×10⁷ CFU to at least about 5×10¹⁰ CFU ART24, at least about 5×10⁸ CFU to at least about 5×10¹⁰ CFU ART24, at least about 5×10⁹ CFU to at least about 5×10¹⁰ CFU ART24. In some embodiments, the pharmaceutical compositions comprise at least about 5×10⁴ CFU ART24. In some embodiments, the pharmaceutical compositions comprise at least about 5×10⁵ CFU ART24. In some embodiments, the pharmaceutical compositions comprise at least about 5×10⁶ CFU ART24. In some embodiments, the pharmaceutical compositions comprise at least about 5×10⁷ CFU ART24. In some embodiments, the pharmaceutical compositions comprise at least about 5×10⁸ CFU ART24. In some embodiments, the pharmaceutical compositions comprise at least about 5×10⁹ CFU ART24.

In some embodiments, the compositions of the present technology contain in a one gram dosage formulation 10⁸ colony forming units (CFU) of viable ART24 bacterium (i.e., vegetative cell) or bacterial spore. In some embodiments, the methods of the present technology involve the administration of about 10⁴ to about 10¹² viable bacteria or spores per day. In some embodiments, the compositions of the present technology are delivered as lyophilized material or powder to be re-suspended for oral delivery or packaged into capsules. The lyophilized material and capsules may be coated for better enteric stability.

An exemplary treatment regimen entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the subject can be administered a prophylactic regime. In some embodiments, compositions of the present technology are administered to a subject once, twice, or three times per day for 10 to 14 days or until the subject is deemed cured of primary disease, not to be at risk for recurrence of primary disease, or not to be at risk for contracting the disease. In some embodiments, administration is paired with a shortened exposure to agents known in the art for the treatment of ulcerative colitis, such as an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and/or filgotinib (GLPG0634, Galapagos and Gilead)), followed by once, twice, or three times daily dosing for 10 to 14 days or until the patient is deemed cured of primary disease or not to be at risk for recurrence of the disease. In some embodiments, methods of prophylaxis comprise administration of compositions of the present technology once, twice, or three times daily for 10 to 14 days or until the patient is deemed not to be at risk of developing the condition in the case of patients known to be at risk for ulcerative colitis.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### VI. Combination Therapy with ART24

In some embodiments, the ART24 strain of the present technology, or spores thereof, may be combined with one or more additional therapies for the prevention or treatment of ulcerative colitis. Additional therapeutic agents include, but are not limited to, one or more additional therapeutic agents selected from the group consisting of: an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib (GLPG0634, Galapagos and Gilead)).

In some embodiments, an additional therapeutic agent is administered to a subject in combination with the ART24 strain of the present technology, or spores thereof, such that a synergistic therapeutic effect is produced. For example, administration of ART24 with one or more additional therapeutic agents for the prevention or treatment of ulcerative colitis will have greater than additive effects in the prevention or treatment of the condition.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. In addition, the combination methods, compositions and formulations are not to be limited to the use of only two agents.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: ART24 Protects Against Ulcerative Colitis In Vivo

### A. Animal Model

This example demonstrates that ART24 protects against ulcerative colitis in an in vivo mouse model.

### Methods

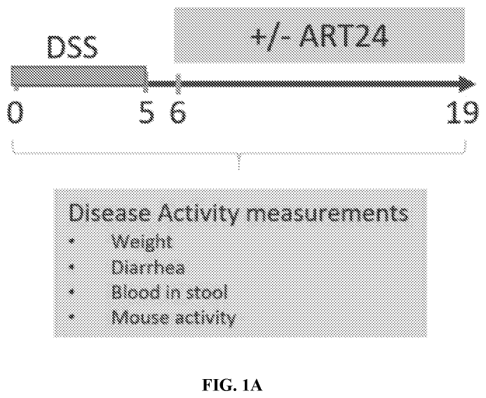

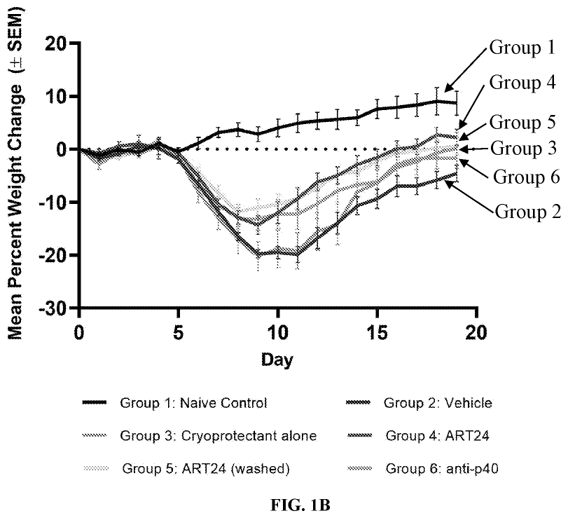

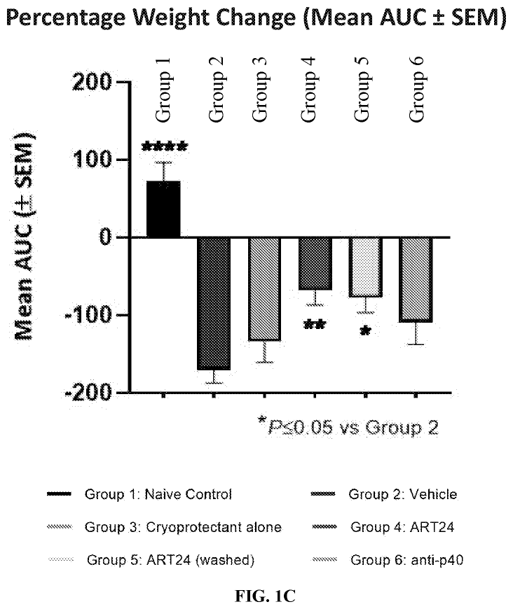

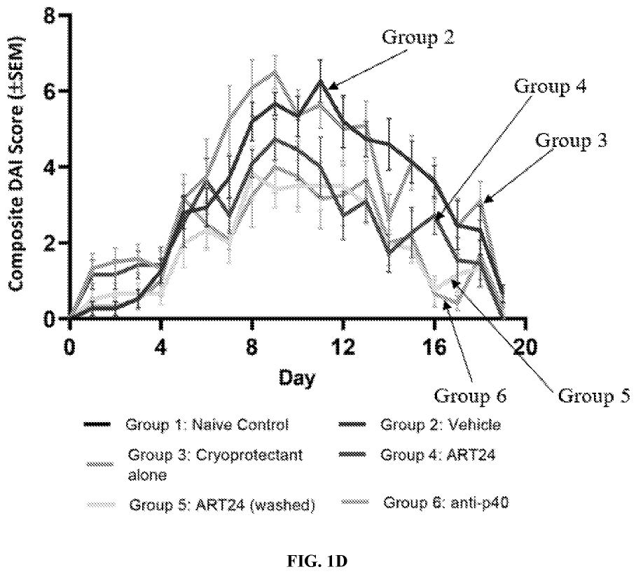

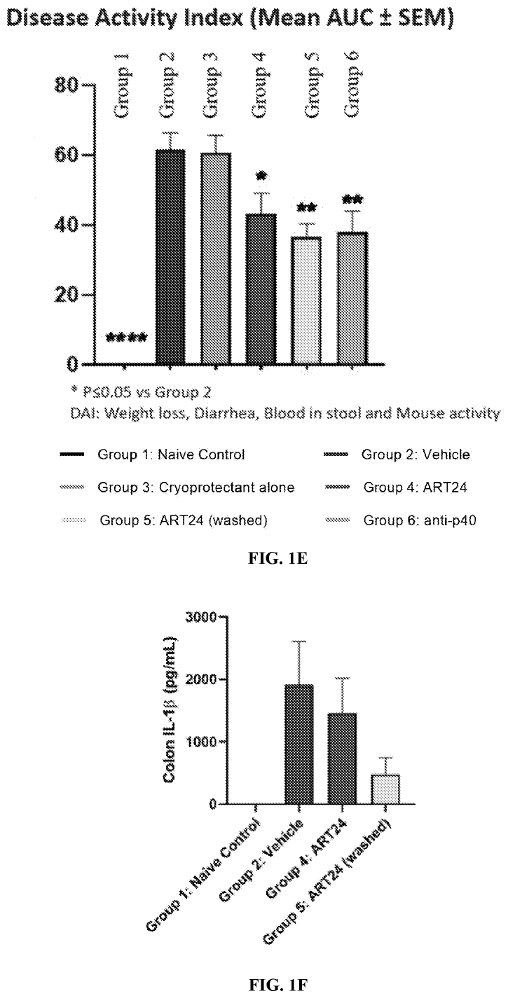

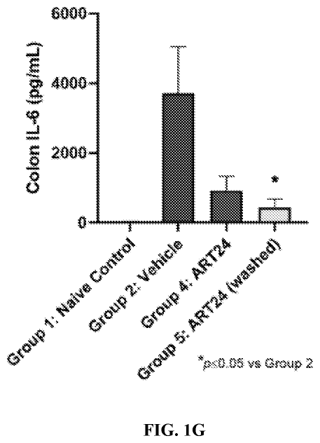

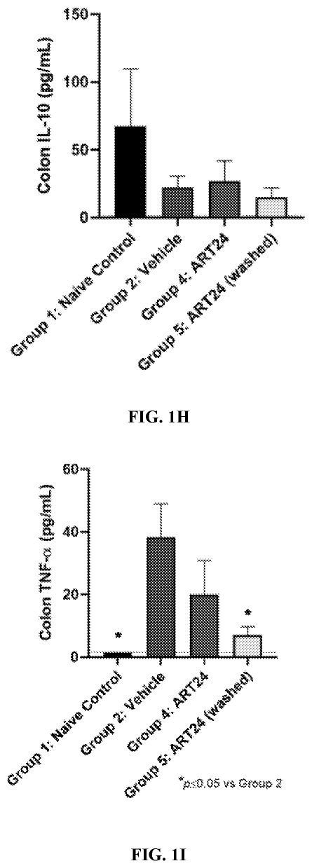

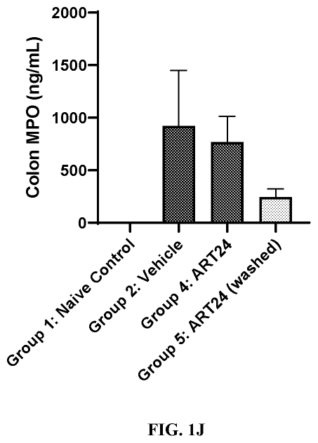

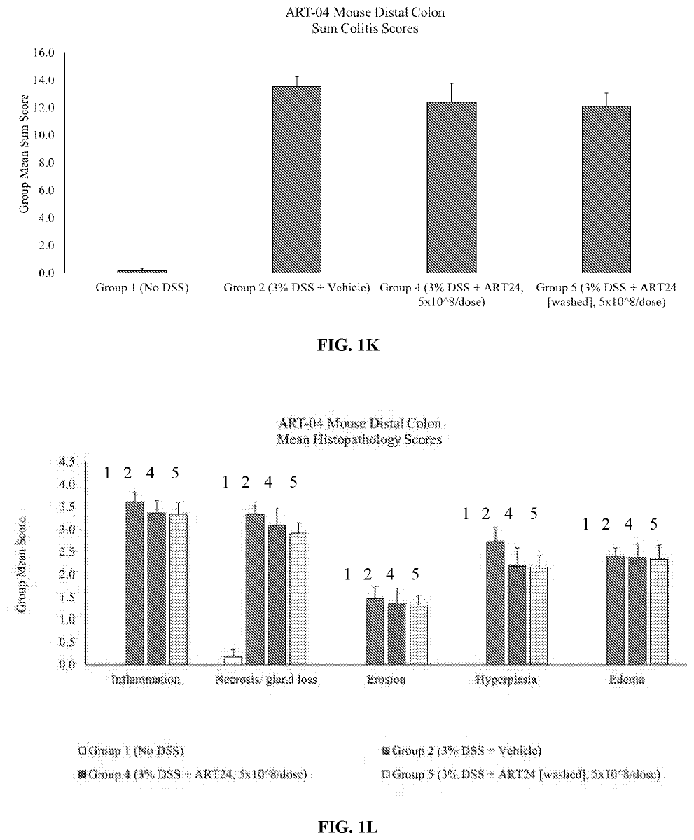

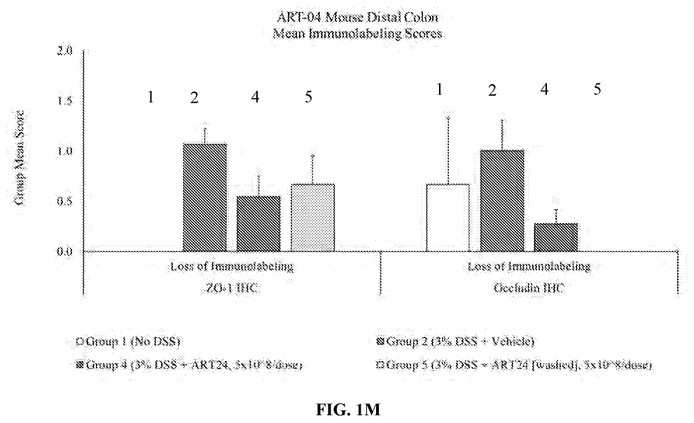

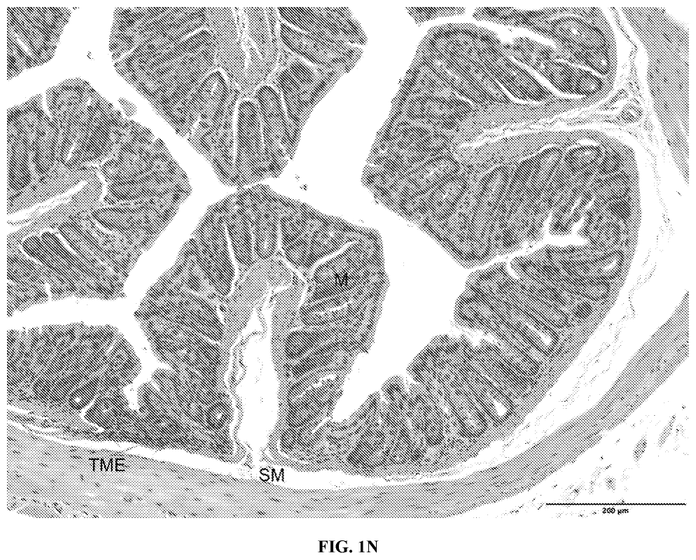

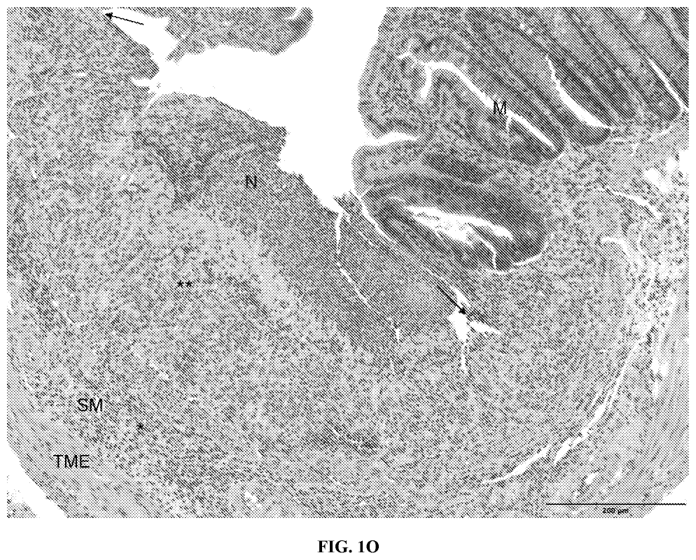

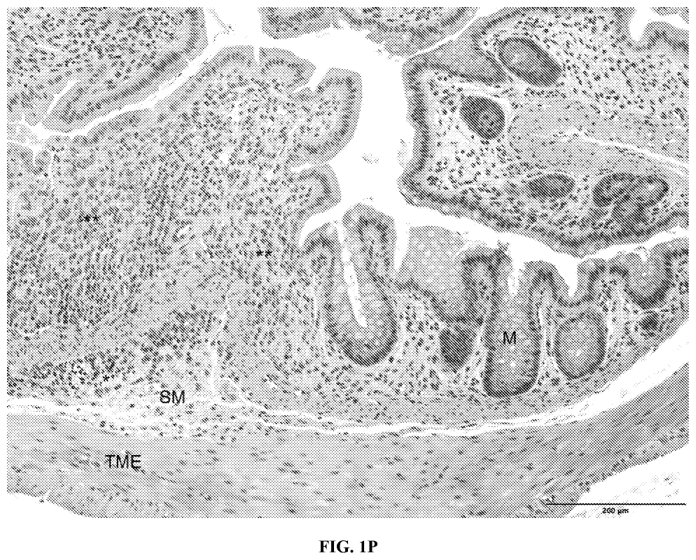

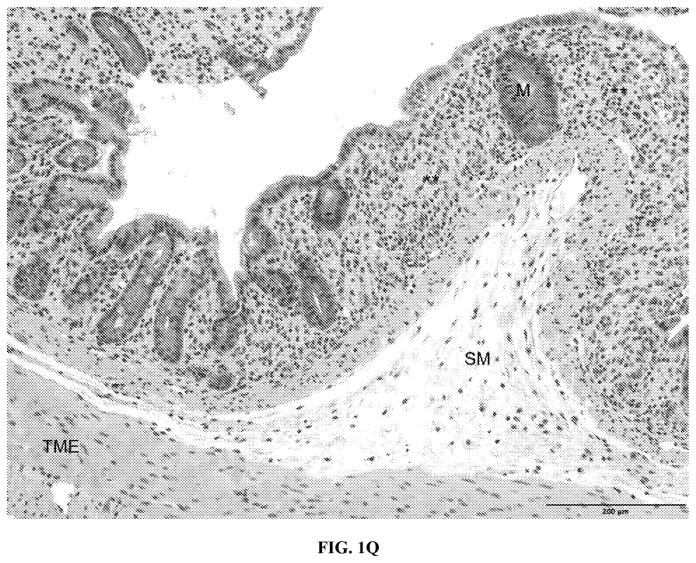

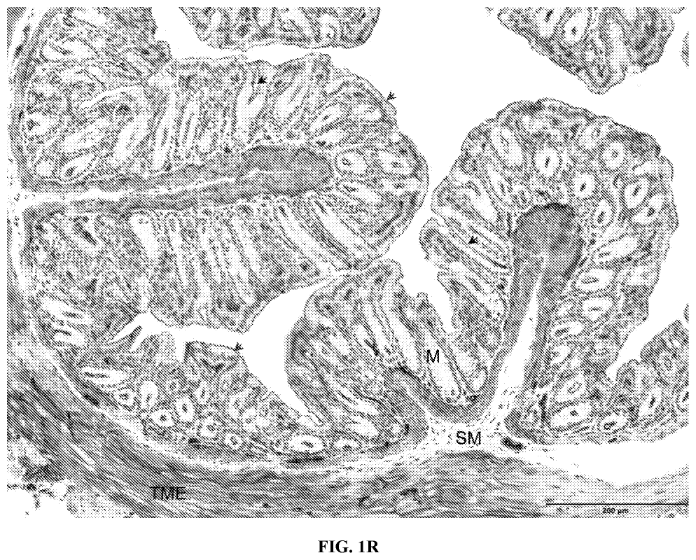

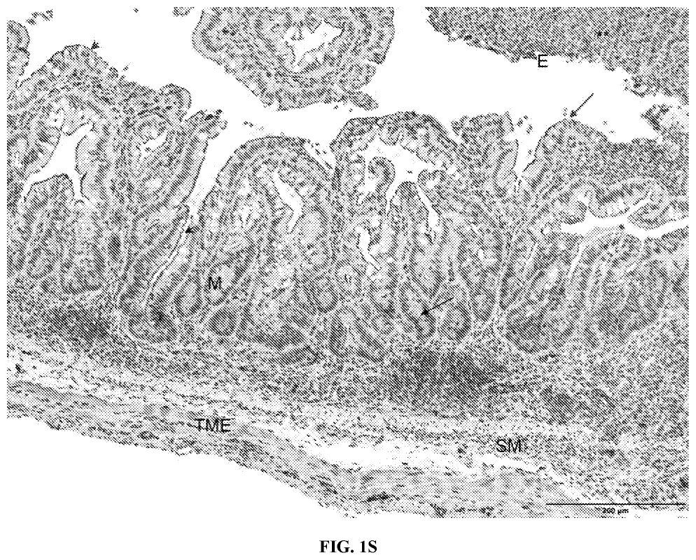

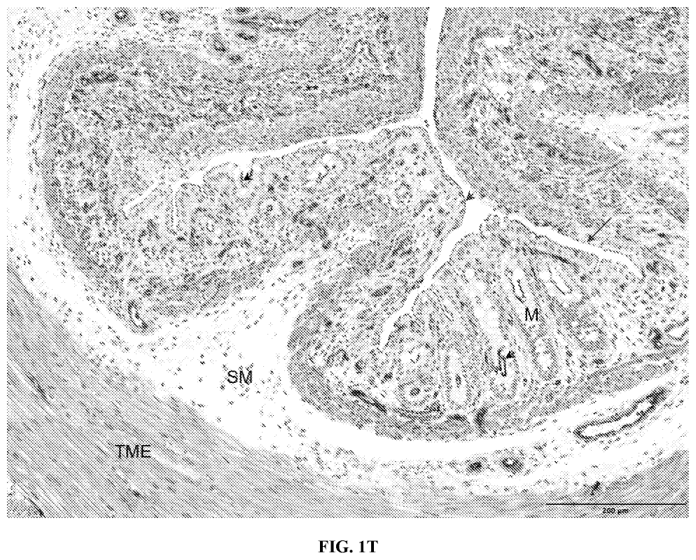

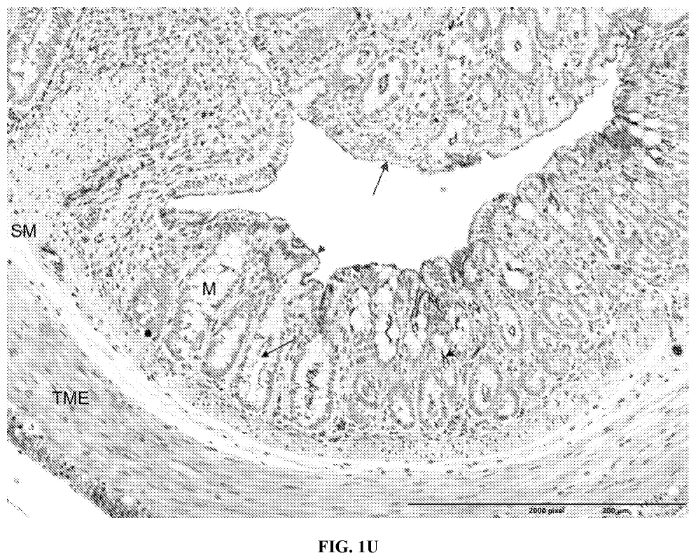

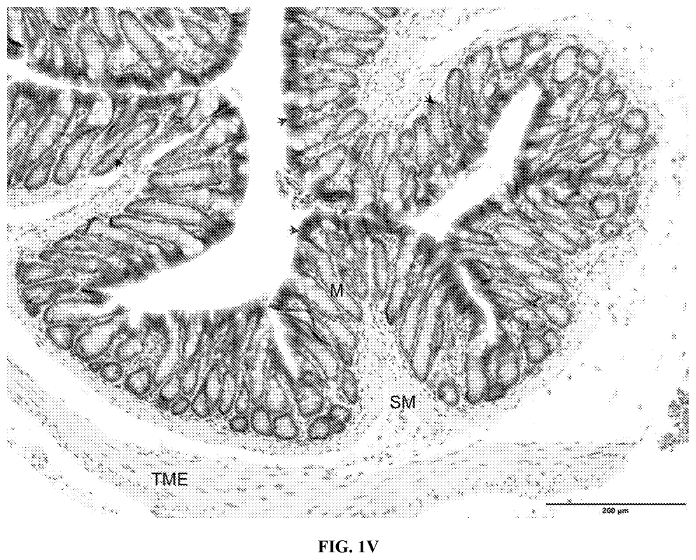

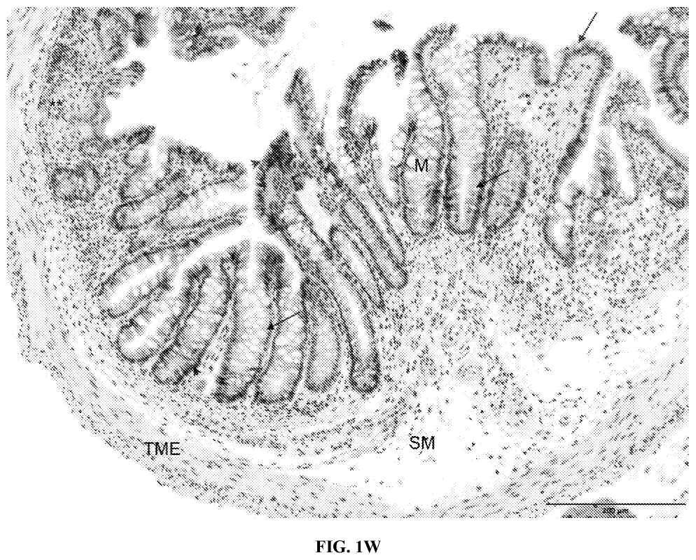

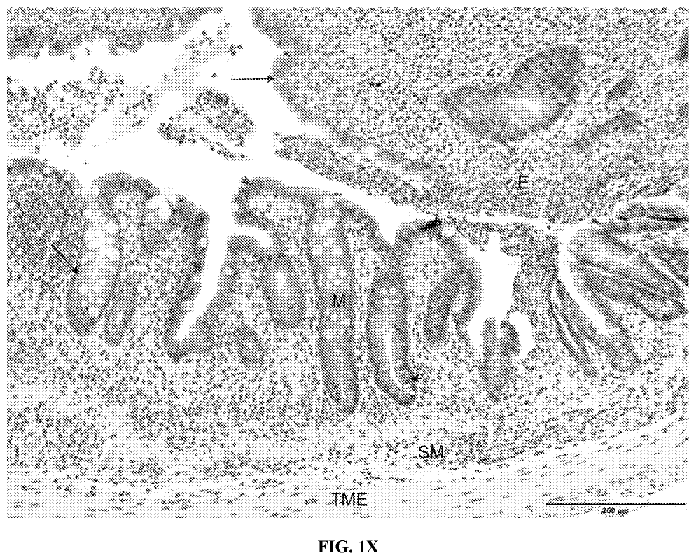

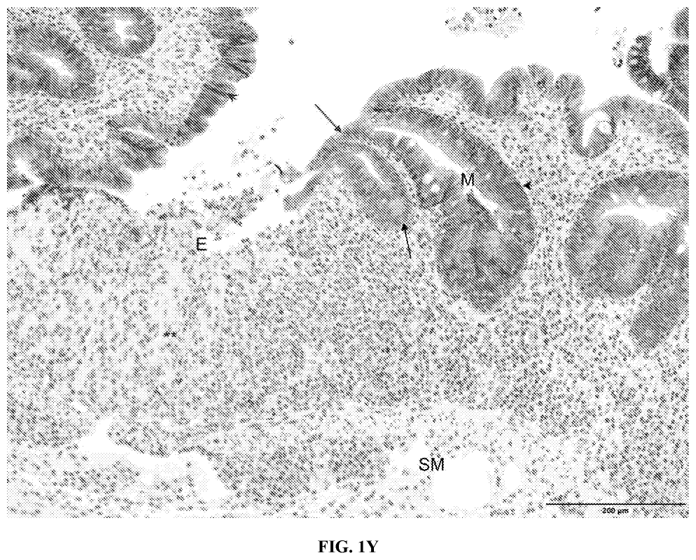

To test the protective effect of ART24 against ulcerative colitis an in vivo model of ulcerative colitis was generated in C57Bl/6 mice. Briefly, 5 groups of mice were exposed to 3% DSS-treated drinking water from Day 0 to Day 5. A sixth group did not receive DSS and served as naïve controls (Group 1). Animals were dosed with Vehicle, cryoprotectant (trehalose) alone (in PBS), ART24, or ART24 washed (aqueous) twice a day (BID) from Days 6 to 19 via oral gavage (Groups 2-5) or with Anti-α40 mAb once a day (QD) on Days 6, 9, 12, 15, as indicated in Table 1 and as shown in FIG. 1A.

TABLE 1
Study Design.

| | | | | | | | | | Terminal |
|---|---|---|---|---|---|---|---|---|---|
| | No. | | | | | | DAI | | Collections |
| Group | Animals | DSS | Treatment | Dose | Schedule | Route | Scoring | Endoscopy | (Day 19) |
| 1 | 6 | — | — | — | — | — | QD | Day | Blood |
| 2 | 15 | Days | Vehicle | — | BID | PO | | 19 | (Serum) |
| | | 0-5 | (PBS) | | Days | | | | Colon |
| 3 | 12 | 3% | Cryo- | — | 6 to 19 | | | | (flash-frozen and |
| | | DSS | protectant | | | | | | formalin-fixed) |
| | | | (trehalose) | | | | | | |
| | | | alone | | | | | | |
| 4 | 12 | | ART24 | 5 × 10⁸/dose | | | | | |
| 5 | 12 | | ART24 | | | | | | |
| | | | (washed) | | | | | | |
| 6 | 12 | | Anti-p40 | 10 mg/kg | QD Days | IP | | | |
| | | | mAb | | 6, 9, 12, | | | | |
| | | | | | 15 & 18 | | | | |

After dosing on Day 12, CFU/mL of ART24 was determined for Groups 4-5. Briefly, 10-100 μL of was serially diluted (10⁰-10⁸ depending on dose used) and spot-plated (10 μL per spot in triplicate) on quartered Soy Yeast Dextrose or BHI agar plates. Plates were incubated at 37° C. under aerobic conditions for 24 hours. The dilution at which approximately 3-30 CFU was observed was counted and CFU/mL determined; dilutions below and above the dilution yielding 3-30 CFU were also be counted. If countable colonies were not observed by 48 hours, plates were removed and “no-growth” was recorded.

All animals were weighed, and monitored (survival, DAI scoring (see Table 2)) daily. Briefly, animals were observed daily for weight loss, diarrhea, blood in stool, and activity. Each of these parameters was individually scored according to the disease activity index (DAI) scoring scheme shown in Table 2, yielding DAI scores with a minimum of 0 (>4.99% body weight loss, no diarrhea, no blood in stool, normal activity) and a maximum of 18 (>30% body weight loss, diarrhea, gross bleeding and moribund). Any animals that lost >30% of their body weight, showed an inability to eat, or were moribund, were euthanized and no samples were collected.

TABLE 2
Disease Activity Index Scoring Scheme

| | Weight | | Blood in | Mouse |
|---|---|---|---|---|
| Score | Loss | Diarrhea | Stool | Activity |
| 0 | 0.0-4.99% | Normal | Not | Normal |
| | | | Present | |
| 1 | 5.0-9.99% | Loose Stools | | |
| 2 | 10.0-14.99% | Loose Stools | Blood | Mild to |
| | | | Observed | moderately |
| | | | | decreased |
| 3 | 15.0-19.99% | Diarrhea | | |
| 4 | >20.0% | Diarrhea | Gross | Stationary |
| | | | Bleeding | until |
| | | | | stimulated |
| 5 | >30.0% | Diarrhea | | Moribund |

At the end of the study (Day 19), all surviving animals from each treatment group were euthanized by C02 inhalation. Blood was collected and processed for serum, then stored at −80° C. Colon samples were removed from the most distal 1.5 cm sections. The middle 2 cm portion was used fro MPO and cytokine ELISA analyses. The tissue samples were snap frozen and stored at −80° C. and formalin sections were stored at room temperature.

Histopathology was carried out as follows. “Distal” colon samples fixed in 10% neutral buffered formalin were trimmed into three transverse sections and embedded together in a single block (six pieces of tissue each with distal away from the label). “Distal” samples represent the most distal portion of the two submitted samples from each animal which exhibited distal colon morphology. Three slides from each block were sectioned at approximately 5 m and stained with hematoxylin and eosin (H&E), or by routine immunohistochemistry (IHC) methods for zonula occludens 1 (ZO-1) or occludin. Glass slides were evaluated by a board-certified veterinary pathologist using light microscopy. Distal colon samples were scored; lesions were scored according to severity 0-5 (0=not present/normal, 1=minimal, 2=mild, 3=moderate, 4=marked, 5=severe). Scored features were added together for each sample to obtain a sum colitis score (Range: 0-25). ZO-1 and occludin immunolabeling were scored for labeling loss in comparison to control samples; only intact epithelium was considered for scoring, i.e., areas of gland loss and/or erosion and squamous metaplasia were not considered when applying the score. Scores ranged from 0-5 as follows: 0=no loss/consistent with most control samples for that colon region, 1=minimal/focal loss, 2=mild loss, 3=moderate loss, 4=marked loss, 5=severe loss.

### Results

Physical Ulcerative Colitis Symptoms. As shown in FIGS. 1B and 1C, there was a reduction in weight loss in groups 4 (ART24 treated) and 5 (washed ART24 treated) mice during portions of the trial. The reduction in weight loss was comparable to that displayed by group 6 mice, who received the positive control anti-p40 treatment. Similarly, FIGS. 1D and 1E demonstrate that treatment groups 4 and 5 had reduced composite DAI scores relative to groups 3 (cryoprotectant alone (trehalose)) and 2 (vehicle) mice.

Inflammatory Markers. Upon euthanasia, colon tissue homogenates were prepared from the Day 19 samples. The colon tissue supernatant was used to determine the protein levels of IL-1β, IL-6, IL-10, and TNF-α in Groups 1, 2, 4, and 5. The mean colon tissue cytokine concentrations±SEM are shown. *p≤0.05. Statistical significance was determined by one-way ANOVA followed by Dunnett's post test for multiple comparisons vs Group 2.

ART24 also ameliorated the inflammatory response associated with ulcerative colitis. Relative to the group 2 vehicle control mice, groups 4 and 5 mice had reduced colonic levels of inflammatory markers IL-1β (FIG. 1F), IL-6 (FIG. 1G), TNF-α (FIG. 1I), and myeloperoxidase (MPO) (FIG. 1J). As shown in FIG. 111, IL-10 (which is an anti-inflammatory cytokine) was not significantly changed by treatment with ART24 at Day 19 during disease resolution.

Histological Morphologic Pathology. Histological analysis of distal colon samples further established the protective effect of ART24 treatment. Administration of DSS to mice produced the expected histologic lesions of subacute inflammation, mucosal necrosis/gland loss, erosions, submucosal edema, and epithelial hyperplasia. Subacute inflammation was characterized by the infiltration of neutrophils, lymphocytes, plasma cells, and macrophages into the mucosa or submucosa. Mucosal necrosis was characterized by colonic gland loss or crypt loss, with an intact surface epithelium; erosion was characterized by the loss of surface and gland epithelium superficial to the muscularis mucosae. Submucosal edema was characterized by the expansion of the submucosa by clear space or pale eosinophilic fluid with lymphatic vascular distention. Reactive epithelial hyperplasia was characterized by gland elongation, reduced goblet cells, and increased mitotic figures in the luminal portion of the colonic gland. Sections of distal colon often exhibited squamous metaplasia in areas of gland loss. In distal colon segments, lesions of ulcerative colitis were absent or minimal in animals not receiving DSS (Group 1). In the distal colon, both ART24 (Group 4) and ART24 (washed; Group 5) were associated with lower sum colitis scores, as seen in FIG. 1M, and generally lower histopathology mean scores, as seen in FIG. 1N, compared to the vehicle-treated samples (Group 2). Representative images of the H&E histopathology are shown for a non-lesioned colons (FIG. 1Q), a group 2 distal colon (FIG. 1R), a group 4 distal colon (FIG. 1S), and a group 5 distal colon (FIG. 1T).

Tight Junction Immunohistochemistry. Two tight junction proteins were evaluated in this study, ZO-1 and occludin. ZO-1 IHC produced moderate to strong intensity contiguous immunolabeling of the glandular epithelia with a primarily apical surface membranous labeling pattern observed. The vascular endothelium, muscularis mucosae, and tunica muscularis externa also exhibited immunolabeling, but these zones were not considered for scoring. Loss of immunolabeling along the gland surface or within glands compared to the no DSS controls (Group 1) was relatively infrequently observed in both colon segments. IHC for occludin produced moderate to strong intensity immunolabeling of glandular epithelia; immunolabeling along membranes between cells of the surface and crypts (between goblet cells) was primarily present. Loss of immunolabeling compared to the no DSS controls (Group 1) was characterized by reduced abundance and/or intensity of immunolabeling in areas of intact epithelium (surface or glandular). In the distal colon samples, DSS administration was generally associated with loss of ZO-1 and occludin immunolabeling compared to the no DSS controls, as shown in FIG. 1P. However, as shown in FIG. 1P, for both tight junction markers, treatment with ART24 (Groups 4 and 5) produced modest (ZO-1) or substantial (occludin) reductions in mean scores. Representative images of the ZO-1 immunohistochemistry are shown for a group 1 distal colon (FIG. 1U), a group 2 distal colon (FIG. 1V), a group 4 distal colon (FIG. 1W), and a group 5 distal colon (FIG. 1X). Representative images for occludin immunohistochemistry are shown for a group 1 distal colon (FIG. 1Y), a group 2 distal colon (FIG. 1Z), a group 4 distal colon (FIG. 1AA), and a group 5 distal colon (FIG. 1AB).

A summary of the histological and immunohistochemistry results are provided as follows: Table 3 summarizes the distal colon results; and Table 4 provides an animal by animal breakdown of the results.

TABLE 3
Mouse Distal Colon Histopathology Summary.
Mouse Distal Colon Histopathology Summary

| | | H&E | | | | | | ZO-1 IHC | Occludin IHC |
|---|---|---|---|---|---|---|---|---|---|
| | | | Necrosis/ | | | | | Loss of | Loss of |
| Group | | Inflammation | gland loss | Erosion | Hyperplasia | Edema | SUM | Immunolabeling | Immunolabeling |
| Group 1 (No DSS) | Mean | 0.00 | 0.17 | 0.00 | 0.00 | 0.00 | 0.17 | 0.00 | 0.67 |
| | SEM | 0.00 | 0.17 | 0.00 | 0.00 | 0.00 | 0.17 | 0.00 | 0.67 |
| Group 2 (3% DSS + | Mean | 3.60 | 3.33 | 1.47 | 2.73 | 2.40 | 13.53 | 1.07 | 1.00 |
| Vehicle) | SEM | 0.21 | 0.19 | 0.26 | 0.30 | 0.19 | 0.70 | 0.15 | 0.31 |
| Group 4 (3% DSS + ART24, | Mean | 3.36 | 3.09 | 1.36 | 2.18 | 2.36 | 12.36 | 0.55 | 0.27 |
| 5 × 10⁸/dose) | SEM | 0.28 | 0.37 | 0.34 | 0.40 | 0.31 | 1.37 | 0.21 | 0.14 |
| Group 5 (3% DSS + ART24 | Mean | 3.33 | 2.92 | 1.33 | 2.17 | 2.33 | 12.08 | 0.67 | 0.00 |
| [washed], 5 × 10⁸/dose) | SEM | 0.26 | 0.23 | 0.19 | 0.24 | 0.31 | 0.96 | 0.28 | 0.00 |

TABLE 4
Mouse Colon Individual Colon Segment Data.
Mouse Colon Individual Colon Segment Data

| | | | H&E | | | | | | ZO-1 IHC | Occludin IHC |
|---|---|---|---|---|---|---|---|---|---|---|
| | | | | Necrosis/ | | | | | Loss of | Loss of |
| Group | Animal | Segment | Inflammation | gland loss | Erosion | Hyperplasia | Edema | SUM | Immunolabeling | Immunolabeling |
| Group 1(No | 1 | Prox | 0 | 0 | 0 | 0 | 0 | 0 | 0 | 0 |
| DSS) | | Dist | 0 | 0 | 0 | 0 | 0 | 0 | 0 | 0 |
| | 2 | Prox | 0 | 0 | 0 | 0 | 0 | 0 | 0 | 0 |
| | | Dist | 0 | 0 | 0 | 0 | 0 | 0 | 0 | 0 |
| | 3 | Prox | 0 | 0 | 0 | 0 | 0 | 0 | 0 | 0 |
| | | Dist | 0 | 0 | 0 | 0 | 0 | 0 | 0 | 0 |
| | 4 | Prox | 0 | 0 | 0 | 0 | 0 | 0 | 0 | 0 |
| | | Dist | 0 | 1 | 0 | 0 | 0 | 1 | 0 | 0 |
| | 5 | Prox | 0 | 0 | 0 | 0 | 0 | 0 | 0 | 0 |
| | | Dist | 0 | 0 | 0 | 0 | 0 | 0 | 0 | 0 |
| | 6 | Prox | 0 | 0 | 0 | 0 | 0 | 0 | 0 | 0 |
| | | Dist | 0 | 0 | 0 | 0 | 0 | 0 | 0 | 4 |
| | | Mean | 0.00 | 0.08 | 0.00 | 0.00 | 0.00 | 0.08 | 0.00 | 0.33 |
| | | SEM | 0.00 | 0.08 | 0.00 | 0.00 | 0.00 | 0.08 | 0.00 | 0.33 |
| Group 2 | 7 | Prox | 2 | 2 | 1 | 2 | 1 | 8 | 0 | 3 |
| (3% DSS + | | Dist | 3 | 3 | 1 | 4 | 2 | 13 | 1 | 3 |
| Vehicle) | 8 | Prox | 1 | 1 | 0 | 1 | 0 | 3 | 0 | 3 |
| | | Dist | 5 | 4 | 3 | 5 | 2 | 19 | 2 | 4 |
| | 9 | Prox | 2 | 1 | 1 | 2 | 1 | 7 | 0 | 1 |
| | | Dist | 4 | 4 | 3 | 1 | 3 | 15 | 0 | 0 |
| | 10 | Prox | 1 | 1 | 0 | 1 | 0 | 3 | 0 | 1 |
| | | Dist | 4 | 3 | 2 | 3 | 2 | 14 | 2 | 1 |
| | 11 | Prox | 1 | 1 | 0 | 1 | 0 | 3 | 0 | 0 |
| | | Dist | 4 | 4 | 1 | 2 | 3 | 14 | 1 | 1 |
| | 12 | Prox | 3 | 3 | 2 | 5 | 1 | 14 | 1 | 2 |
| | | Dist | 5 | 4 | 1 | 1 | 3 | 14 | 1 | 1 |
| | 13 | Prox | 2 | 1 | 1 | 3 | 1 | 8 | 1 | 1 |
| | | Dist | 3 | 2 | 1 | 2 | 2 | 10 | 1 | 1 |
| | 14 | Prox | 1 | 1 | 0 | 0 | 0 | 2 | 0 | 1 |
| | | Dist | 4 | 4 | 1 | 3 | 2 | 14 | 1 | 1 |
| | 15 | Prox | 0 | 0 | 0 | 0 | 0 | 0 | 0 | 1 |
| | | Dist | 2 | 2 | 1 | 2 | 2 | 9 | 1 | 2 |
| | 16 | Prox | 1 | 1 | 0 | 1 | 1 | 4 | 0 | 1 |
| | | Dist | 4 | 4 | 2 | 3 | 3 | 16 | 1 | 1 |
| | 17 | Prox | 1 | 1 | 0 | 0 | 0 | 2 | 0 | 0 |
| | | Dist | 3 | 3 | 0 | 4 | 2 | 12 | 0 | 0 |
| | 18 | Prox | 0 | 0 | 0 | 1 | 0 | 1 | 0 | 0 |
| | | Dist | 3 | 3 | 0 | 4 | 2 | 12 | 1 | 0 |
| | 19 | Prox | 1 | 1 | 0 | 2 | 1 | 5 | 1 | 0 |
| | | Dist | 4 | 4 | 3 | 2 | 4 | 17 | 2 | 0 |
| | 20 | Prox | 2 | 1 | 1 | 3 | 1 | 8 | 2 | 1 |
| | | Dist | 3 | 3 | 1 | 2 | 1 | 10 | 1 | 0 |
| | 21 | Prox | 2 | 1 | 1 | 3 | 1 | 8 | 1 | 0 |
| | | Dist | 3 | 3 | 2 | 3 | 3 | 14 | 1 | 0 |
| | | Mean | 2.47 | 2.20 | 0.97 | 2.20 | 1.47 | 9.30 | 0.73 | 1.00 |
| | | SEM | 0.26 | 0.25 | 0.18 | 0.25 | 0.21 | 0.98 | 0.13 | 0.20 |
| Group 4 | 34 | Prox | 1 | 1 | 1 | 2 | 1 | 6 | 0 | 0 |
| (3% DSS + | | Dist | 3 | 3 | 0 | 2 | 2 | 10 | 1 | 1 |
| ART24, | 35 | Prox | 1 | 0 | 0 | 0 | 0 | 1 | 0 | 0 |
| 5 × 10⁸/dose) | | Dist | 4 | 4 | 2 | 4 | 2 | 16 | 1 | 1 |
| | 36 | Prox | 2 | 1 | 1 | 2 | 0 | 6 | 0 | 0 |
| | | Dist | 2 | 2 | 1 | 1 | 2 | 8 | 0 | 0 |
| | 37 | Prox | 1 | 1 | 0 | 0 | 0 | 2 | 0 | 1 |
| | | Dist | 4 | 3 | 1 | 5 | 3 | 16 | 1 | 0 |
| | 38 | Prox | 0 | 0 | 0 | 0 | 0 | 0 | 0 | 0 |
| | | Dist | 2 | 1 | 0 | 1 | 2 | 6 | 0 | 0 |
| | 39 | Prox | 0 | 0 | 0 | 0 | 0 | 0 | 0 | 0 |
| | | Dist | 2 | 1 | 0 | 1 | 0 | 4 | 0 | 0 |
| | 40 | Prox | 1 | 1 | 0 | 2 | 1 | 5 | 1 | 0 |
| | | Dist | 4 | 4 | 2 | 2 | 3 | 15 | 1 | 0 |
| | 41 | Prox | 2 | 1 | 1 | 2 | 1 | 7 | 1 | 0 |
| | | Dist | 4 | 4 | 2 | 1 | 4 | 15 | 0 | 0 |
| | 43 | Prox | 1 | 1 | 0 | 1 | 0 | 3 | 0 | 0 |
| | | Dist | 4 | 4 | 3 | 3 | 3 | 17 | 0 | 0 |
| | 44 | Prox | 1 | 1 | 0 | 1 | 0 | 3 | 1 | 1 |
| | | Dist | 4 | 4 | 1 | 2 | 3 | 14 | 0 | 0 |
| | 45 | Prox | 2 | 1 | 1 | 2 | 0 | 6 | 2 | 1 |
| | | Dist | 4 | 4 | 3 | 2 | 2 | 15 | 2 | 1 |
| | | Mean | 2.23 | 1.91 | 0.86 | 1.64 | 1.32 | 7.95 | 0.50 | 0.27 |
| | | SEM | 0.30 | 0.32 | 0.21 | 0.27 | 0.28 | 1.23 | 0.14 | 0.10 |
| Group 5 | 46 | Prox | 2 | 2 | 2 | 4 | 1 | 11 | 0 | 1 |
| (3% DSS + | | Dist | 4 | 4 | 2 | 2 | 3 | 15 | 0 | 0 |
| ART24 [washed], | 47 | Prox | 1 | 1 | 1 | 1 | 1 | 5 | 0 | 0 |
| 5 × 10⁸/dose) | | Dist | 4 | 3 | 1 | 2 | 3 | 13 | 0 | 0 |
| | 48 | Prox | 1 | 1 | 1 | 2 | 1 | 6 | 0 | 1 |
| | | Dist | 3 | 3 | 2 | 2 | 3 | 13 | 0 | 0 |
| | 49 | Prox | 1 | 0 | 0 | 0 | 0 | 1 | 0 | 0 |
| | | Dist | 4 | 4 | 2 | 3 | 3 | 16 | 2 | 0 |
| | 50 | Prox | 2 | 2 | 2 | 4 | 1 | 11 | 0 | 2 |
| | | Dist | 3 | 3 | 1 | 2 | 3 | 12 | 0 | 0 |
| | 51 | Prox | 1 | 1 | 0 | 0 | 0 | 2 | 0 | 0 |
| | | Dist | 1 | 1 | 0 | 1 | 0 | 3 | 1 | 0 |
| | 52 | Prox | 2 | 1 | 1 | 3 | 1 | 8 | 2 | 2 |
| | | Dist | 3 | 3 | 1 | 1 | 3 | 11 | 3 | 0 |
| | 53 | Prox | 2 | 2 | 2 | 3 | 2 | 11 | 2 | 0 |
| | | Dist | 3 | 3 | 1 | 2 | 3 | 12 | 0 | 0 |
| | 54 | Prox | 2 | 1 | 1 | 2 | 1 | 7 | 0 | 0 |
| | | Dist | 3 | 2 | 1 | 3 | 1 | 10 | 0 | 0 |
| | 55 | Prox | 2 | 1 | 1 | 3 | 1 | 8 | 0 | 1 |
| | | Dist | 4 | 3 | 2 | 4 | 2 | 15 | 1 | 0 |
| | 56 | Prox | 3 | 2 | 2 | 4 | 0 | 11 | 0 | 2 |
| | | Dist | 4 | 3 | 2 | 2 | 1 | 12 | 0 | 0 |
| | 57 | Prox | 1 | 1 | 0 | 2 | 1 | 5 | 0 | 1 |
| | | Dist | 4 | 3 | 1 | 2 | 3 | 13 | 1 | 0 |
| | | Mean | 2.50 | 2.08 | 1.21 | 2.25 | 1.58 | 9.63 | 0.50 | 0.42 |
| | | SEM | 0.23 | 0.22 | 0.15 | 0.24 | 0.23 | 0.86 | 0.18 | 0.15 |

Accordingly, these results show that ART24 protects against the physical manifestations and pathology of ulcerative colitis and reduces the pro-inflammatory signals associated with the disease in an in vivo model, and is effective in methods for preventing, reducing the risk of, or treating ulcerative colitis.

### B. Human Subjects

Human subjects diagnosed as having or suspected to have ulcerative colitis and presently displaying one or more symptoms and/or pathologies of ulcerative colitis, are recruited using selection criteria known and accepted in the art.

### Methods of Prevention and Treatment

Subjects are administered ART24 at a dosage and frequency commensurate with the stage and severity of disease. In some embodiments a compound is administered once daily, once weekly, or once monthly. In some embodiments, a compound is administered multiple times daily, weekly, or monthly.

To demonstrate methods of prevention and treatment in humans, subjects are administered ART24 prior to or subsequent to the development of symptoms and/or pathologies of ulcerative colitis and assessed for reversal of symptoms/pathologies or attenuation of expected symptoms/pathologies using methods known in the art.

### Results

It is expected that ART24 will induce reversal of symptoms and/or pathologies of ulcerative colitis in human subjects. These results will show that ART24 is useful and effective for the prevention and treatment of such a disorder.

### Example 2: ART24 Protects Against Ulcerative Colitis In Vivo as Demonstrated by Endoscopy and Colon Weight/Length Ratio

This example prophetically demonstrates that ART24 is protective against ulcerative colitis, as assessed by endoscopy measurements, stool consistency, and colon weight/length ratio.

### Methods

To test the protective effect of ART24 against ulcerative colitis, as assessed by endoscopy, stool consistency, and colon weight/length ratio, an in vivo model of ulcerative colitis will be generated according to the methods of Example 1.

Animals will undergo video endoscopy on Day 19 to assess ulcerative colitis severity. Images will be captured from each animal at the most severe region of disease identified during endoscopy, and stool consistency is scored during endoscopy using the parameters defined in Table 5. Ulcerative colitis severity is scored using a 0-4 scale (0=normal; 1=loss of vascularity; 2=loss of vascularity and friability; 3=friability and erosions; 4=ulcerations and bleeding). Following endoscopy on Day 19, all surviving animals from each treatment group are euthanized by CO₂ inhalation. Blood is collected and processed for serum, and stored at −80° C. The colon is excised, measured, rinsed, weighed, and trimmed to the most distal 5 cm in length. The most distal and proximal 1.5 cm sections are each placed in formalin for subsequent histological evaluation. All snap frozen tissue samples are stored at −80° C. and formalin sections are stored at room temperature.

TABLE 5
Stool Consistency

| Score | Description |
|---|---|
| 0 | Normal, well-formed pellet |
| 1 | Loose stool, soft, staying in shape |
| 2 | Loose stool, abnormal form with excess moisture |
| 3 | Watery or diarrhea |
| 4 | Bloody diarrhea |

### Results

It is expected that ART24 will induce an improvement in endoscopy and stool consistency measurements, and in colon weight/length ratios. These results will show that ART24 is useful and effective for the prevention and treatment of ulcerative colitis.

### Example 3: ART24 is within EFSA Antibiotic Susceptibility Guidelines and does not Contain Virulence Factors or Pathogenicity Islands

As demonstrated by Table 6, ART24 is within the European Food Safety Authority (EFSA) guidelines. The minimum inhibitory concentration (MIC) of the listed antimicrobials is expressed as μg/mL. Additional studies following EFSA testing recommendations were performed on lyophilized ART24 lots CO-33-10A2 and CO-33-12A2 and Research and Master Cell Banks CO-33-ART24 BHI and CO-33-ART24 SYD, and those results are shown in Table 7.

TABLE 6
ART24 within EFSA antibiotic susceptibility guidelines

| Antimicrobial | ART24 MIC (μg/mL) | EFSA guidelines |
|---|---|---|
| Ampicillin | 0.25 | n.r. |
| Vancomycin | 0.5-1 | 4 |
| Gentamicin* | ≤0.5 | 4 |
| Kanamycin* | ≤2 | 8 |
| Streptomycin | 4 | 8 |
| Erythromycin | 0.12 | 4 |
| Clindamycin | 0.5-1 | 4 |
| Tetracycline | 4 | 8 |
| Chloroamphenicol | 4 | 8 |
| Neomycin | ≤0.5 | — |
| Penicillin | 0.12-0.5 | — |
| Quinupristin-dalfopristin | 4 | — |
| Linezolid | 0.5 | — |
| Trimethoprim | <0.12 | — |
| Ciprofloxacin* | ≤0.25 | — |
| Rifampicin | 0.5 | — |

Average results from N = 6.
*No growth with the lowest concentration in the plate.

TABLE 7
ART24 within EFSA antibiotic susceptibility guidelines

| | MIC in mg/L (interpretation) for ART24 for all 4 sources^{a} | | | | | | | |
|---|---|---|---|---|---|---|---|---|
| | CO-33 ART 24 | | CO-33 ART 024 | | CO-33- | | CO-33- | |
| Antimicrobial | BHI | | SYD | | 10A2 | | 12A2 | |
| Ampicillin | ≤0.03 | (S) | ≤0.03 | (S) | ≤0.03 | (S) | ≤0.03 | (S) |
| Chloramphenicol | 2 | (S) | 2 | (S) | 2 | (S) | 2 | (S) |
| Ciprofloxacin | ≤0.03 | (S) | ≤0.03 | (S) | ≤0.03 | (S) | ≤0.03 | (S) |
| Clindamycin | 0.25 | (S) | 0.25 | (S) | 0.12 | (S) | 0.25 | (S) |
| Erythromycin | 0.12 | (S) | 0.12 | (S) | 0.06 | (S) | 0.06 | (S) |
| Fidaxomicin | 4 | | 4 | | 2 | | 4 | |
| Gentamicin | ≤0.06 | (S) | ≤0.06 | (S) | ≤0.06 | (S) | ≤0.06 | (S) |
| Kanamycin | ≤0.5 | (S) | ≤0.5 | (S) | ≤0.5 | (S) | ≤0.5 | (S) |
| Linezolid | 0.5 | | 0.5 | | 0.25 | | 0.5 | |
| Metronidazole | >64 | | >64 | | >64 | | >64 | |
| (aerobic) | | | | | | | | |
| Metronidazole | 64 | | 64 | | 64 | | 64 | |
| (anaerobic) | | | | | | | | |
| Neomycin | ≤0.12 | | ≤0.12 | | ≤0.12 | | ≤0.12 | |
| Penicillin | ≤0.03 | (S) | 0.06 | (S) | ≤0.03 | (S) | ≤0.03 | (S) |
| Quinupristin- | 2 | | 2 | | 1 | | 2 | |
| dalfopristin | | | | | | | | |
| Rifampicin | 0.25 | (S) | 0.25 | (S) | 0.25 | (S) | 0.25 | (S) |
| Streptomycin | 1 | (S) | 2 | (S) | 1 | (S) | 1 | (S) |
| Tetracycline | 2 | (S) | 2 | (S) | 2 | (S) | 2 | (S) |
| Trimethoprim | <0.25 | | <0.25 | | ≤0.25 | | <0.25 | |
| Vancomycin | 0.5 | (S) | ≤0.25 | (S) | ≤0.25 | (S) | ≤0.25 | (S) |

^{a}Categorical interpretations using CLSI M45 (2015) breakpoint criteria, as available. MIC interpretations for kanamycin and streptomycin used European Food Safety Authority cutoff values (2012).

Briefly, material from 3-5 colonies of ART24 were selected and suspended in 4 mL of MRD to obtain a cell concentration of ˜1×10⁸ CFU/mL. From this suspension 46 μL was transferred in to 23 mL of 90% Iso-Sensitest Broth (CM0473, Oxoid) containing 10% BHI broth to end with a final inoculum concentration of 5×10⁵ CFU/mL. The wells of the prefabricated 96-well plates were filled with 100 μL of the final suspension. The plates were sealed with an AeraSeal (A9224, Sigma Aldrich) sealing film (to facilitate growth of the aerobic strains. The plates were then incubated for 24 hr at 37° C. The results are recorded after 24 hours as the lowest concentration of the antibiotic to inhibit visible growth. The prefabricated panels VetMIC Lact-1 & Lact-2 (SVA, Uppsala, Sweden) comprising the antibiotics ampicillin, vancomycin, gentamicin, kanamycin, streptomycin, erythromycin, clindamycin, tetracycline, chloramphenicol, neomycin, penicillin, quinupristin-dalfopristin, linezolid, trimethoprim, ciprofloxacin and rifampicin were used.

The absence of antibiotic resistance in the strain of the present technology precludes the risk of horizontal transfer of antibiotic resistance.

Moreover, searches conducted with KEGG, RAST SEED Viewer, IslandViewer 4 and ACT comparisons to known Bacillus species that produce toxins, etc., did not reveal any virulence factors or pathogenicity islands in the genomes of ART24. Accordingly, these results demonstrate the probiotic safety of the ART24 in that it lacks the propensity to confer resistance to commensals and/or pathogens.

### Example 4: Freeze-Drying (Lyophilization) to Preserve ART24

This example demonstrates that ART24 is useful in compositions and methods wherein the strain is freeze-dried (or lyophilized).

2×SG Sporulation media: Difco nutrient broth 16.0 g/L; KCl, 2.0 g/L; MgSO₄·7H₂O, 0.5 g/L. Adjust the pH to 7.0, autoclave. After autoclaving and cooling add 1 M Ca(NO₃)₂·4H₂O, 1.0 ml/L; 0.1M MnCl₂·4H₂O, 1.0 ml/L; 1 mM FeSO₄·7H₂O, 1.0 ml/L; 50% (w/v) glucose, 2.0 ml/L.

Spore preparation method: Inoculate 50 ml of BHI (1.10493.0500, Merck) medium with cells from a fresh colony of ART24. Grow aerobically in flasks 24 hours at 37° C. with shaking (orbital platform shaker) at 200 rpm. Dilute 1/200 into 1 L 2×SG medium in an appropriate vessel and grow at 37° C. with shaking at 200 rpm. Check samples for spores daily using light microscopy. Phase contrast microscopy is ideal. After 2-3 days >90% of the population should have sporulated. Pellet cells at 9000 rpm for 20 minutes in a centrifuge if possible, keeping temperatures low, otherwise for small quantities a benchtop centrifuge will suffice. Wash and centrifuge the spores with ice-cold water 2-3 times to remove residual nutrients and lyse remaining vegetative cells. Resuspend the spore pellet in 10 mg/ml lysozyme solution and incubate shaking at 37° C. for 1 hour. Wash and centrifuge the spores with ice-cold water 4-6 times to remove residual nutrients and lyse remaining vegetative cells. Freeze dry 3 ml aliquots of spores over 24 hr period in a VirTis Advantage Wizard freeze dryer and store in sealed freeze-drying vials at room temperature for long-term storage.

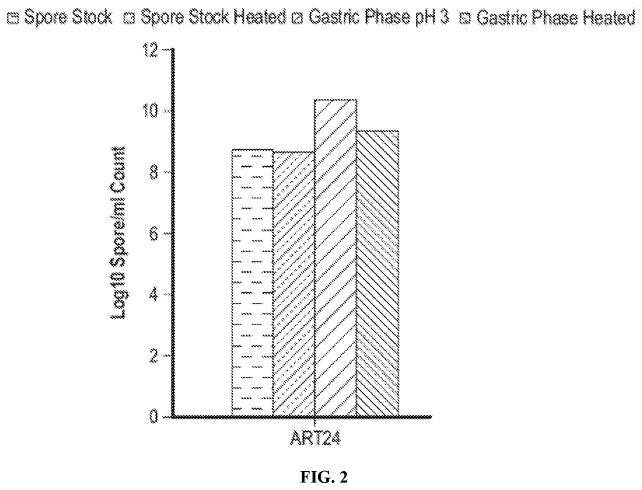

Simulated gastric fluid was prepared based on the SGF outlined by the Infogest COST action containing 37.3 g/L KCl, 68 g/L KH₂PO₄, 84 g/L NaHCO₃, 117 g/L NaCl, 30.5 g/L (NH₄)CO₃, 44.1 g/L CaCl₂) and 2 mL of a 20,000 U/mL solution. As shown in FIG. 2, spores of ART24 survive when exposed to simulated gastric fluid.

As shown in Table 8, spores of ART24 achieve 100% survivability after freeze drying and resuspension.

To test the robustness of the cells, the strain was freeze-dried. Briefly, overnight cultures of ART24 were grown according to methods known in the art and re-suspended in 15% w/v trehalose solution. Cell counts were performed.

The cultures were freeze-dried for 22 hours, after which they were re-suspended in an equivalent volume of water lost. Cell counts were performed. Table 8 shows the survivability results.

TABLE 8
Survivability of ART24 after freeze drying (lyophilization)

| | Pre FD | Post FD |
|---|---|---|
| | (log) | (Log) |
| ART24 | 8.2 | 8.9 |

Accordingly, these results demonstrate that ART24 is useful in compositions and methods wherein the strain is freeze-dried (or lyophilized).

### Example 5: ART24 Protects Against Ulcerative Colitis In Vivo (Twice Daily Dosing)

This example demonstrates that ART24 protects against ulcerative colitis in an in vivo mouse model.

### Methods

To test the protective effect of ART24 against ulcerative colitis an in vivo model of ulcerative colitis was generated in C57Bl/6 mice. Briefly, colitis was induced in 90 C57Bl/6 male mice by exposure to 3% DSS-treated drinking water between Day 0 and Day 5. A group of six (6) animals did not receive DSS and served as naïve controls (Group 1). Groups 2-4 were dosed via oral gavage (PO) with either vehicle (sterile saline) or two different doses of ART24 (highest 5×10⁸ CFU and lowest 5×10⁴ CFU) twice per day (BID) from Days 6-12. Groups 5 and 6 were dosed PO twice per day (BID) from Days 6-12. The first dose (AM) consisted of one or two doses of ART24 (highest 1×10⁹ CFU per day and lowest 1×10⁵ CFU per day). The second dose (PM) was an equal volume of vehicle each day. The animals in Group 7 received anti-p40 antibody (in PBS) every 3 days beginning on Day 6 (doses on Days 6, 9, and 12) via IP injection. The dosing volume for Groups 2-6 was 0.1 mL flat per animal and 0.1 mL/20 g for Group 7. The study design is provided in Table 9.

TABLE 9
Study Design.

| | | | | Dose | | | | | Terminal |
|---|---|---|---|---|---|---|---|---|---|
| | No | | | (total | | | DAI | | Collections |
| Group | Animals | DSS | Treatment | CFU/dose) | Schedule | Route | Scoring | Endoscopy | (Day 12) |
| 1 | 6 | — | — | — | — | — | Days | Day 12 | Whole blood (RO) |
| 2 | 20 | Days | Vehicle | — | BID | PO | 0-12 | | serum |
| | | 0-5 | (saline) | | Days | | | | Colon |
| 3 | 15 | 3% | ART24 | 5 × 10⁴ | 6 to 12 | | | | Distal 2 cm- snap |
| 4 | 15 | DSS | | 5 × 10⁸ | | | | | frozen |
| 5 | 15 | | ART24 | 1 × 10⁵ | BID | | | | Next most distal 2 cm- |
| 6 | 15 | | | 1 × 10⁹ CFU | (1 active | | | | fixed |
| | | | | | dose | | | | Colon Contents |
| | | | | | AM + 1 | | | | (expressed without |
| | | | | | vehicle | | | | flushing) |
| | | | | | dose | | | | Flash frozen |
| | | | | | PM) | | | | Cecum contents |
| | | | | | Days | | | | collection (flash |
| | | | | | 6 to 12 | | | | frozen)- expressed |
| 7 | 10 | | Anti-p40 | 10 mg/kg | QD Days | IP | | | without flushing |
| | | | mAb | | 6, 9, 12 | | | | |

All animals were weighed and monitored for composite DAI scoring (see Table 10) daily.

TABLE 10
Disease Activity Index Scoring Scheme

| Score | Weight Loss | Diarrhea | Blood in Stool |
|---|---|---|---|
| 0 | 0.0-4.99% | Normal | Not Present |
| 1 | 5.0-9.99% | | |
| 2 | 10.0-14.99% | Loose Stools | Blood Observed |
| 3 | 15.0-19.99% | | |
| 4 | >20.0% | Diarrhea | Gross Bleeding |
| 5 | >30.0% | | |

Animals underwent video endoscopy on Day 12 to assess colitis severity. Images were captured from each animal at the most severe region of disease identified during endoscopy, and colitis severity and stool consistency was scored during endoscopy using the parameters defined in Tables 11-12.

TABLE 11
Colitis Severity

| Score | Description |
|---|---|
| 0 | Normal |
| 1 | Loss of vascularity |
| 2 | Loss of vascularity and friability |
| 3 | Friability and erosions |
| 4 | Ulcerations and bleeding |

TABLE 12
Stool Consistency

| Score | Description |
|---|---|
| 0 | Normal, well-formed pellet |
| 1 | Loose stool, soft, staying in shape |
| 2 | Loose stool, abnormal form with excess moisture |
| 3 | Watery or diarrhea |
| 4 | Blood diarrhea |

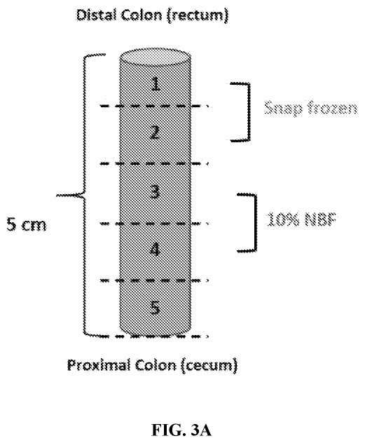

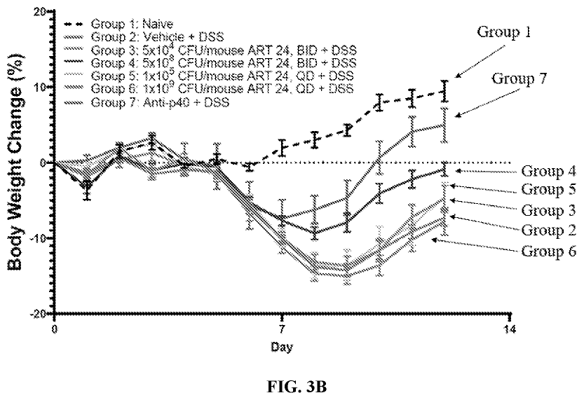

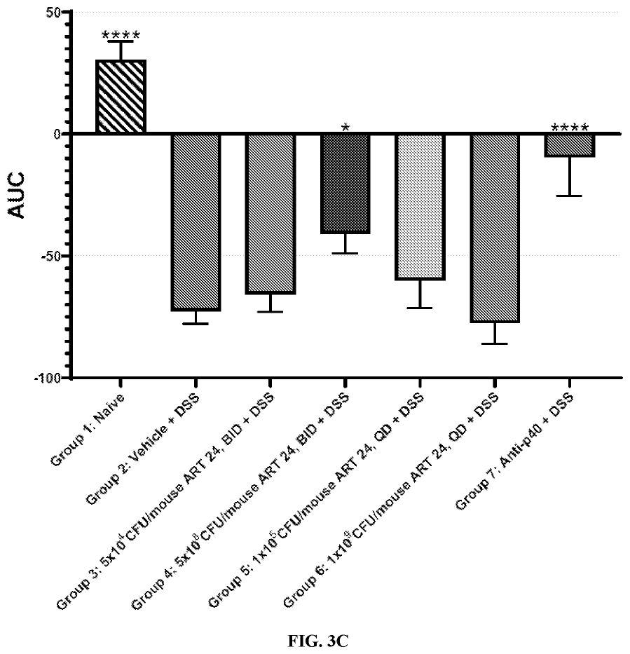

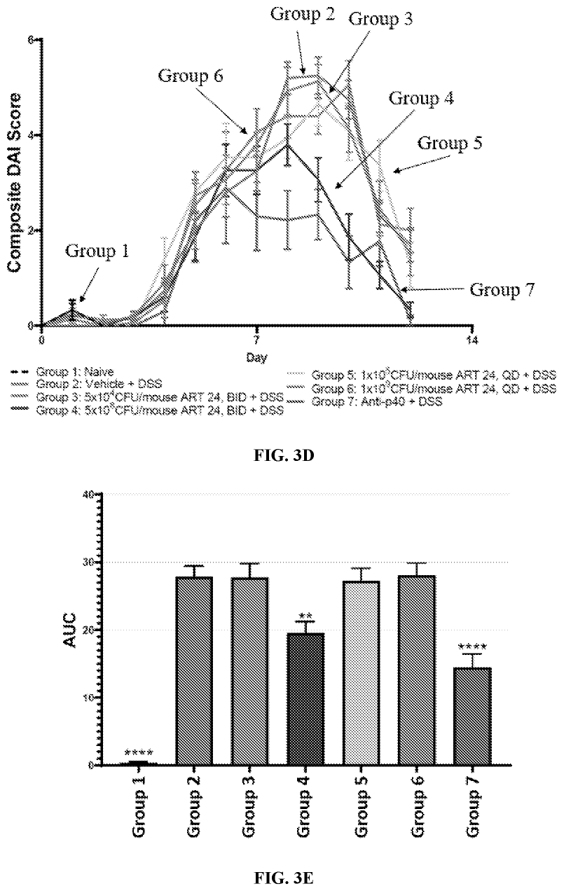

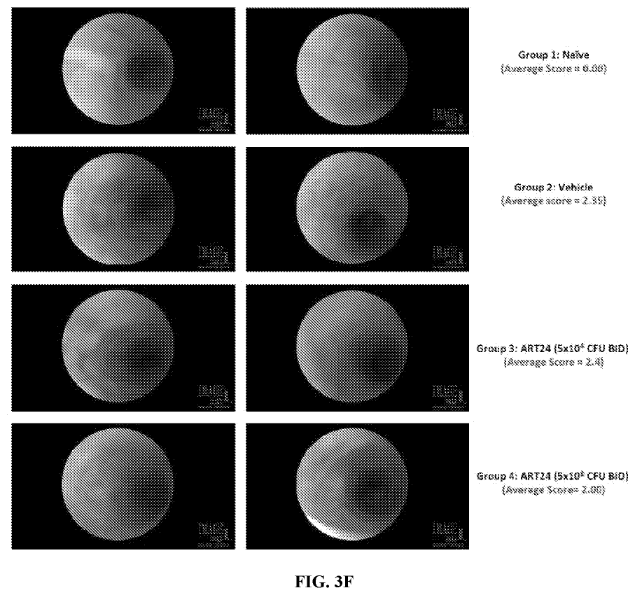

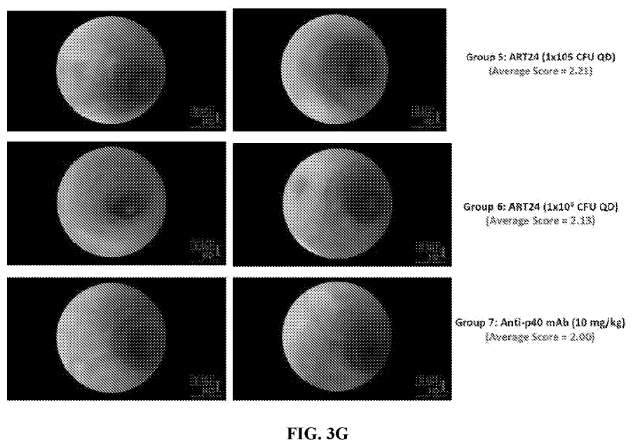

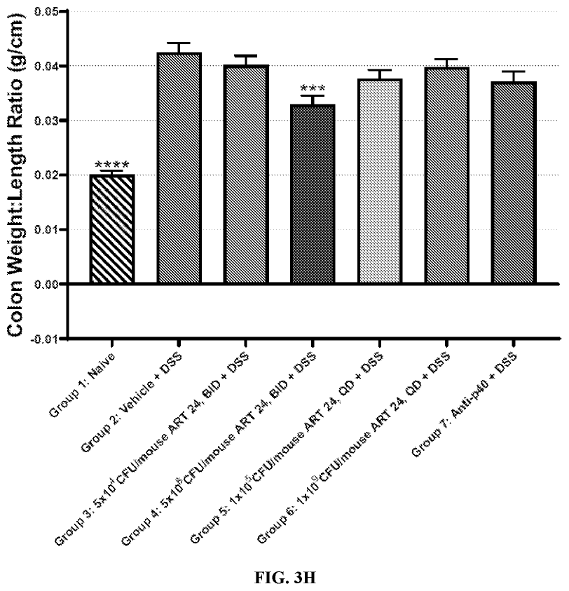

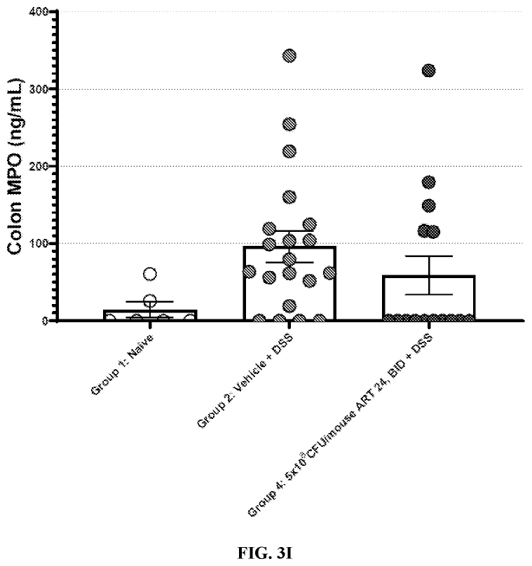

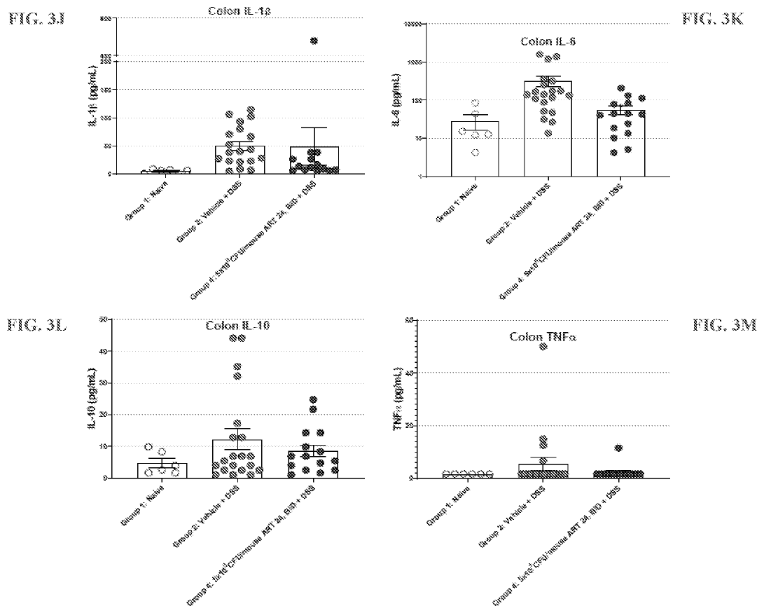

Following endoscopy on Day 12, all surviving animals from each treatment group were sacrificed by C02 inhalation for terminal organ collection, as follows. All animals were euthanized by C02 inhalation following endoscopy on Day 12. Blood was collected via RO bleed (terminal) and processed for serum. Serum was stored at −80° C. The colon was excised and measured. Colon contents were collected without flushing, weighed, and snap frozen for possible downstream analysis. The colon was then rinsed, weighed, and the most distal 5 cm was isolated. The most distal 2 cm of colon (from the rectum, sections 1 and 2) was weighed and snap frozen. The next most distal 2 cm of colon (sections 3 and 4) was fixed in 10% NBF for possible downstream histopathology. In addition, cecum contents were also collected without rinsing, weighed and snap frozen. All snap frozen tissue samples were stored at −80° C. until used for selected endpoint analysis. The details of the colon tissue collection are shown in FIG. 3A. Colon tissue collected at euthanasia from a subset of animals (Groups 1-2 and 4) were used to quantify MPO and cytokine colonic levels.

Animals. Male C57Bl/6 mice (n=96; 6-8 weeks) with an average starting body weight (±SEM) of 20.28±0.15 g were obtained from Charles River Laboratories (Wilmington, Massachusetts). Animals were acclimatized prior to study commencement. During this period, the animals were observed daily in order to reject any that presented in poor condition.

Housing. The study was performed in animal rooms provided with HEPA filtered air at a temperature of 70±5° F. and 50%±20% relative humidity. Animals were housed in groups of 6-20 per cage. Animal rooms were set to maintain a minimum of 12 to 15 air changes per hour. The room was on an automatic timer for a light/dark cycle of 12 hours on and 12 hours off with no twilight. Bed-OCobs® or equivalent bedding was used. Animals were provided with enviro-dri, shepherd shacks, and nestlets. Cages, tops, and water bottles were washed with a commercial detergent and allowed to air dry. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number, and treatment group was used to mark all cages. The temperature and relative humidity was recorded during the study, and the records retained.

Diet. Animals were fed with LabDiet 5053 rodent diet and water was provided ad libitum. No foodbased enrichment was provided.

Animal randomization and allocations. Animals were randomized into seven (7) groups at the start of the study: one (1) group of six (6) mice, one (1) group of ten (10) mice, one (1) group of 20 mice, and four (4) groups of 15 mice each. Each animal was identified by an ear punch corresponding to an individual number. A cage card was used to identify each cage and was marked with the study number, treatment group number, and animal numbers.

Disease induction. Colitis was induced in all animals in Groups 2-7 via addition of 3% DSS (MP Biomedicals) to the drinking water from Days 0 to 5. Animals in Group 1 did not receive DSS in the drinking water and served as naïve controls.

Test articles and formulation. Details regarding the test articles are provided in Table 13.

TABLE 13
Test Articles and Formulation

| Test Article | Anti-p40 | ART24 |
|---|---|---|
| Name of the Test | Mouse anti- | ART24 (lyophilized |
| | IL-12 p40 | bacteria) |
| Article | (Clone: C17.8) | |
| | (BioXCell) | |
| Storage | 4° C. | −80° C. |
| conditions | | |
| Vehicle | PBS | Sterile saline |
| Dose | 10 mg/kg | 1 × 10⁵ and 1 × 10⁹ CFU |
| | | per animal per day |
| Frequency | Q3D; Days 6, 9, | BID, Days 6 to 12 (Groups |
| and duration | and 12 | 5 and 6 will receive an |
| of dosing | | active dose in AM and 1 |
| | | vehicle dose in PM) |
| Route and method | IP; 0.1 mL/20 g | PO; 0.1 mL flat per animal |
| of administration | | |

Body weight and survival. Animals were observed daily (weight, morbidity, survival, presence of diarrhea and/or blood stool) in order to assess possible differences among treatment groups and/or possible toxicity resulting from the treatments.

Animal health observations and disease activity index (DAI). Animals were observed daily for signs of adverse animal health (e.g., visual inspection of movement, lethargy, normal mouse behaviors), as well as survival, in order to assess possible differences among treatment groups and/or possible toxicity resulting from the treatments. Animals were also observed daily for weight loss, diarrhea, and blood in stool. Each of these parameters were individually scored according to the disease activity index (DAI) scoring scheme shown in Table 10, yielding DAI scores with a minimum of 0 (<4.99% body weight loss, no diarrhea, no blood in stool) and a maximum of 13 (>30% body weight loss, diarrhea, and gross bleeding).

Endoscopy. Each mouse underwent video endoscopy on Day 12 using a small animal endoscope (Karl Storz Endoskope, Germany), under isoflurane anesthesia, prior to euthanasia. During each endoscopic procedure, still images as well as video were recorded to evaluate the extent of colitis and the response to treatment. Additionally, an attempt was made to capture an image from each animal at the most severe region of disease identified during endoscopy. Colitis severity was scored using the parameters defined in Table 11. Additionally, stool consistency was scored during endoscopy using the parameters defined in Table 12.

Supportive care and euthanasia criteria. Animals in excess of 15% weight loss were administered 1 mL saline daily. Animals in excess of 20% weight loss were administered 1 mL saline twice daily and were provided with water-softened food pellets. Water-softened food pellets were administered to all animals if they were needed by any one animal. No supportive care was administered while the animals were being exposed to DSS (Days 0-5). Any animal that lost >30% of its body weight, showed an inability to eat, or was moribund, was euthanized. Animals that were found dead or were euthanized prior to scheduled termination days did not undergo terminal collections.

Sacrifice and sample collection. All surviving animals were euthanized by CO2 inhalation following endoscopy on Day 12. Blood and colon samples were collected at sacrifice from animals, as follows.

Blood. Blood was collected via terminal retro-orbital (RO) bleed into Serum Separator tubes. Blood was centrifuged and serum was collected and stored at −80° C. Serum was split into two (2) tubes, 0.1 mL in the 1st aliquot, and the remainder in the 2^{nd} aliquot.

Colon contents. Colon contents from all animals (expressed without flushing) were collected, weighed, and flash frozen. Contents were stored at −80° C. until shipment or analysis.

Cecum contents. Cecum contents from all animals (expressed without flushing) were collected, weighed, and flash frozen. Contents were stored at −80° C. until shipment or analysis.

Colon. The colon was excised and measured. Colon contents were collected without flushing, weighed, and snap frozen for possible downstream analysis. The colon was then rinsed, weighed, and the most distal 5 cm was isolated. The most distal 2 cm of colon (from the rectum, sections 1 and 2) was weighed and snap frozen. The next most distal 2 cm of colon (sections 3 and 4) was fixed in 10% NBF for possible downstream histopathology. The most proximal section was discarded. All snap frozen tissue samples were stored at −80° C. until shipment to the study sponsor or used for selected endpoint analysis. The details of the colon tissue collection are shown in FIG. 3A.

Survival. Animal deaths were evaluated during the course of the study. In this model, animal deaths are commonly attributable to severe colitis or perforation of the colon during endoscopy. In this study, two (2) animals were found dead.

Colon tissue processing for immunoassays. Colon tissue from a subset of animals was homogenized for downstream analysis of myeloperoxidase (MPO) and cytokine analysis, as described below. Specifically, PBS with protease inhibitor was added to flash-frozen colon samples collected from animals in Groups 1-2 and 4 and samples were homogenized in a bead beater. Samples were centrifuged (1000×g; 5 minutes) to pellet insoluble material. Supernatant was transferred to a fresh tube centrifuged again (10,000×g; 10 minutes). Supernatant was aliquoted (two aliquots for below assays and remainder; if possible), flash-frozen, and stored at −80° C. until use for assays described below.

Colon MPO analysis. Colon homogenate supernatant samples from animals in Groups 1-2 and 4 were assessed for MPO by commercially available ELISA (Hycult Biotech, HK210-01) according to manufacturer's instructions. Samples were diluted 1:20 for analysis and were run in duplicate. A standard curve was generated and evaluated for fitness. Sample values that were out-of-range (OOR) on the low end of the curve were entered as 0; if on the high end of the curve, they were entered as the highest standard value.

Multiplex cytokine analysis of colon tissue homogenate. Colon tissue homogenate supernatants from animals in Groups 1-2 and 4 were analyzed for protein levels of mouse inflammatory mediators: IL-1β, IL-6, IL-10, and TNFα using a multiplex system (MAGPIX, EMD Millipore) according to manufacturer's instructions. Samples were undiluted and were run in single replicate. A standard curve was generated and evaluated for fitness for each analyte on the panel. Two quality controls were analyzed for each plate, one high and one low, each of which was evaluated on the MAGPIX software (Luminex Corp). Sample values that were OOR on the low end of the curve were entered as 50% of the lowest standard; if on the high end of the curve, they were entered as the highest standard value.

Outcome evaluation. Study endpoints were body weight change, DAI composite scores, colitis severity and stool consistency scores, colon MPO levels, and colon tissue cytokine levels (IL-1β, IL-6, IL-10, and TNFα).

Statistical analyses. Data were evaluated using one-way ANOVA with Holm-Šídák's multiple comparisons test to compare all groups to the vehicle/DSS control group.

Colitis severity and stool consistency scores were evaluated using Kruskal-Wallis test with Dunn's multiple comparisons test to compare all groups to the vehicle/DSS control group. All statistical analyses were performed using GraphPad Prism 8.4.3 (La Jolla, CA). Statistical significance was achieved when p<0.05.

### Results

Body weight. Animals were weighed daily for the duration of the study and the percent body weight change relative to Day 0 (FIG. 3B) was calculated. In order to determine the statistical significance of overall differences in mean body weight and percent body weight change between groups, the area under the curve (AUC) was calculated using the trapezoidal rule transformation and is shown in FIG. 3C.

Animals in which colitis was induced by exposure to 3% DSS demonstrated mean body weight loss from Days 5 to 9. Body weight increased thereafter, returning to baseline or near-baseline for the majority of groups by study end. Overall mean body weight loss was significantly lower (p<0.0001) in naïve animals (Group 1) in comparison to those treated with DSS and vehicle. Significantly decreased overall mean body weight loss was observed in animals treated with antip40 (Group 7) (p<0.0001) and in animals treated with 5×10⁸ CFU/mouse ART24 BID (Group 4) (p<0.05) when compared to animals treated with vehicle and DSS.

Composite DAI scores. Animals were observed daily for weight loss, diarrhea, and blood in stool. Each of these parameters was individually scored on a daily basis according to the disease activity index (DAI) scoring scheme shown in Table 10, yielding DAI scores with a minimum of 0 (<4.99% body weight loss, no diarrhea, no blood in stool) and a maximum of 12 (>20% body weight loss, diarrhea, and gross bleeding). Composite DAI scores over time are shown in FIG. 3D. In order to determine the statistical significance of overall differences in mean DAI score between groups, the area under the curve (AUC) was calculated using the trapezoidal rule transformation and is shown in FIG. 3E.

Mean composite DAI scores in animals in which colitis was induced increased between Days 4 and 10 and decreased thereafter until sacrifice. Overall mean composite DAI scores were significantly lower (p<0.0001) in naïve animals as compared to those treated with vehicle and DSS. No major differences in overall composite DAI scores were observed in animals treated BID with 5×10⁴ CFU/mouse, QD with 1×10⁵ CFU/mouse, or QD with 1×10⁹ CFU/mouse of ART 24 (Groups 3, 5, and 6, respectively) as compared to those treated with vehicle and DSS. In contrast, significant decreases in overall mean composite DAI scores were observed in animals treated BID with 5×10⁸ CFU/mouse ART24 (p<0.01), as well as in those treated with anti-p40 (p<0.0001), relative to animals treated with vehicle and DSS.

Endoscopy. Animals underwent endoscopy on Day 12 in order to assess colonic inflammation. Colitis was scored visually on a five-point scale that ranges from 0 for normal to 4 for severe ulceration (Table 11). Stool consistency was likewise scored visually on a five-point scale that ranges from 0 for normal to 4 for bloody diarrhea (Table 12). Representative endoscopy images are shown in FIGS. 3F-3G.

Colon measurements at euthanasia. All surviving animals were euthanized on Day 12. The colon was excised, weighed, and measured at this time, and the colon weight:length ratio was calculated (FIG. 3H).

As shown in FIG. 3H, animals in which colitis was induced demonstrated increased mean colon weight:length ratios as compared to naïve animals, and mean colon weight:length ratio was significantly lower (p<0.0001) in naïve animals as compared to animals treated with vehicle and DSS. Animals treated with 5×10⁸ CFU/mouse ART24 BID displayed significantly decreased (p<0.001) colon weight:length ratio when compared to animals treated with vehicle and DSS.

Colon tissue MPO. Flash-frozen colon samples from a subset of animals (Groups 1-2 and 4) were homogenized. Resultant supernatant was analyzed for MPO by commercially available ELISA, and the results are shown in FIG. 3I. Increased mean colon MPO was observed in animals in which disease was induced as compared to naïve animals. Mean colon MPO was reduced in animals treated with 5×10⁸ CFU/mouse ART24 BID.

Colon tissue homogenate cytokines. Colon tissue collected at euthanasia from a subset of animals (Groups 1-2 and 4) was homogenized in PBS plus protease inhibitor, and the resulting homogenate was centrifuged to obtain clear supernatant containing the total tissue protein. This colon tissue supernatant was used to determine the protein concentrations of IL-1β, IL-6, IL-10, and TNFα, shown in FIGS. 3J-3M.

All cytokines were elevated in disease-induced Groups 2 and 4 as compared to naïve animals. Animals treated with 5×10⁸ CFU/mouse ART24 BID displayed lower mean levels of IL-6, and IL-10 when compared to animals treated with vehicle and DSS; this difference was statistically significant at the p<0.05 level in the case of IL-6 levels. Mean TNFα levels were nearly universally below the lower limit of detection, aside from a few outlier animals in the groups in which disease was induced. No major differences in mean colon IL-1β were observed when comparing animals treated with vehicle and DSS and those treated with ART24.

In summary, these results show that ART24 protects against the physical manifestations and pathology of ulcerative colitis and reduces the pro-inflammatory signals associated with the disease in an in vivo model, and is effective in methods for preventing, reducing the risk of, or treating ulcerative colitis.

### BIOLOGICAL DEPOSITS

The Applicant requests that a sample of the deposited microorganisms should be made available only to an expert approved by the Applicant.

Bacillus amyloliquefaciens strain ART24 deposited with the National Collection of Industrial Food and Marine Bacteria (NCIMB Ltd.) (International Depositary Authority), Ferguson Building, Craibstone Estate, Bucksburn, Aberdeen, AB21 9YA, Scotland, on 21 Jun. 2018, under NCIMB Accession Number 43088.

The deposit was made according to the Budapest treaty on the international recognition of the deposit of microorganisms for the purposes of patent procedure.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology.

Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

What is claimed is:

1. A method for treating or preventing ulcerative colitis in a subject in need thereof, the method comprising administering to the subject a therapeutically effective amount of a pharmaceutical composition comprising bacterial strain ART24 (NCIMB Accession No. 43088).

2. The method of claim 1, wherein the ulcerative colitis is ulcerative proctitis, proctosigmoiditis, left-sided colitis, or pancolitis.

3. The method of claim 1 or claim 2, wherein the bacterial strain is lyophilized.

4. The method of any one of claims 1-3, wherein the bacterial strain is in the form of a spore.

5. The method of any one of claims 1-3, wherein the bacterial strain is in vegetative form.

6. The method of any one of claims 1-3, wherein the bacterial strain is a mixture of the strain in the form of a spore and in the vegetative form.

7. The method of any one of claims 1-6, wherein the bacterial strain is formulated as a dietary supplement.

8. The method of any one of claims 1-6, wherein the bacterial strain is formulated as an edible product.

9. The method of claim 8, wherein the edible product further comprises a carrier, vehicle, or excipient.

10. The method of claim 8, wherein the edible product further comprises a fermented food product, soybean, mushroom, mung bean, locus bean, rice, or extracts thereof.

11. The method of claim 10, wherein the soybean is fermented soybean or fermented soybean paste.

12. The method of claim 11, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

13. The method of any one of claims 1-12, wherein the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

14. The method of any one of claims 1-13, wherein the pharmaceutical composition is administered enterally.

15. The method of any one of claims 1-14, further comprising separately, sequentially, or simultaneously administering one or more additional agents to the subject.

16. The method of claim 15, wherein the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

17. The method of any one of claims 1-16, wherein the subject is a human.

18. The method of any one of claims 1-17, wherein the treatment comprises one or more of a reduction in weight loss, an improvement in disease activity index (DAI) scores, or reduced colonic levels of inflammatory markers.

19. The method of claim 18, wherein the colonic inflammatory markers comprise one or more of IL-1β, IL-6, TNF-α, or myeloperoxidase (MPO).

20. Use of a composition in the preparation of a medicament for treating or preventing ulcerative colitis in a subject in need thereof, wherein the composition comprises bacterial strain ART24 (NCIMB Accession No. 43088.

21. The use of claim 20, wherein the ulcerative colitis is ulcerative proctitis, proctosigmoiditis, left-sided colitis, or pancolitis.

22. The use of claim 20 or claim 21, wherein the bacterial strain is lyophilized.

23. The use of any one of claims 20-22, wherein the bacterial strain is in the form of a spore.

24. The use of any one of claims 20-22, wherein the bacterial strain is in vegetative form.

25. The use of any one of claims 20-22, wherein the bacterial strain is a mixture of the strain in the form of a spore and in the vegetative form.

26. The use of any one of claims 20-25, wherein the bacterial strain is formulated as a dietary supplement.

27. The use of any one of claims 20-25, wherein the bacterial strain is formulated as an edible product.

28. The use of claim 27, wherein the edible product further comprises a carrier, vehicle, or excipient.

29. The use of claim 27, wherein the edible product further comprises a fermented food product, soybean, mushroom, mung bean, locus bean, rice, or extracts thereof.

30. The use of claim 29, wherein the soybean is fermented soybean or fermented soybean paste.

31. The use of claim 30, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

32. The use of any one of claims 20-31, wherein the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

33. The use of any one of claims 20-31, wherein the pharmaceutical composition is formulated for enteral administration.

34. The use of any one of claims 19-33, further comprising separately, sequentially, or simultaneously administering one or more additional agents to the subject.

35. The use of claim 34, wherein the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

36. The use of any one of claims 20-35, wherein the subject is a human.

37. The use of any one of claims 20-36, wherein the treatment comprises one or more of a reduction in weight loss, an improvement in disease activity index (DAI) scores, or reduced colonic levels of inflammatory markers.

38. The use of claim 37, wherein the colonic inflammatory markers comprise one or more of IL-1β, IL-6, TNF-α, or myeloperoxidase (MPO).

39. A composition used for treating or preventing ulcerative colitis in a subject in need thereof, wherein the composition comprises bacterial strain ART24 (NCIMB Accession No. 43088.

40. The composition for use of claim 39, wherein the ulcerative colitis is ulcerative proctitis, proctosigmoiditis, left-sided colitis, or pancolitis.

41. The composition for use of claim 39 or claim 40, wherein the bacterial strain is lyophilized.

42. The composition for use of any one of claims 39-41, wherein the bacterial strain is in the form of a spore.

43. The composition for use of any one of claims 39-41, wherein the bacterial strain is in vegetative form.

44. The composition for use of any one of claims 39-41, wherein the bacterial strain is a mixture of the strain in the form of a spore and in the vegetative form.

45. The composition for use of any one of claims 39-44, wherein the bacterial strain is formulated as a dietary supplement.

46. The composition for use of any one of claims 39-44, wherein the bacterial strain is formulated as an edible product.

47. The composition for use of claim 46, wherein the edible product further comprises a carrier, vehicle, or excipient.

48. The composition for use of claim 46, wherein the edible product further comprises a fermented food product, soybean, mushroom, mung bean, locus bean, rice, or extracts thereof.

49. The composition for use of claim 48, wherein the soybean is fermented soybean or fermented soybean paste.

50. The composition for use of claim 49, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

51. The composition for use of any one of claims 39-50, wherein the ulcerative colitis comprises one or more of diarrhea with blood and/or mucous, abdominal pain and cramping, rectal pain, rectal bleeding, urgency to defecate, inability to defecate despite urgency, weight loss, tachycardia, anemia, bowel distension, fatigue, fever, or failure to grow (in children)

52. The composition for use of any one of claims 39-50, wherein the pharmaceutical composition is formulated for enteral administration.

53. The composition for use of any one of claims 39-52, further comprising separately, sequentially, or simultaneously administering one or more additional agents to the subject.

54. The composition for use of claim 53, wherein the one or more additional agents is selected from the group consisting of an anti-inflammatory drug in the 5-aminosalicylate family (e.g., sulfasalazine, mesalamine, balsalazide, and olsalazine), a corticosteroid (e.g., prednisone, budesonide), an immunomodulator (e.g., 6-mercaptopurine, azathioprine, methotrexate) a TNF-α antibody (e.g., infliximab, adalimumab, golimumab), an integrin α4β7 inhibitors (e.g., vedolizumab), an IL-12/23 antibody (e.g., ustekinumab), a calcineurin inhibitor (e.g., cyclosporine, tacrolimus), a Janus kinase (JAK) inhibitor (e.g., tofacitinib), ABT-494 (AbbVie), and filgotinib.

55. The composition for use of any one of claims 39-54, wherein the subject is a human.

56. The composition for use of any one of claims 39-55, wherein the treatment comprises one or more of a reduction in weight loss, an improvement in disease activity index (DAI) scores, or reduced colonic levels of inflammatory markers.

57. The composition for use of claim 56, wherein the colonic inflammatory markers comprise one or more of IL-1β, IL-6, TNF-α, or myeloperoxidase (MPO).
